(19) United States
(12) Patent Application Publication
(10) Pub. No.: US 2024/0409553 A1
(43) Pub. Date: Dec. 12, 2024

(54) HETEROCYCLIC NLRP3 INHIBITORS

(71) Applicant: Hoffmann-La Roche Inc., Little Falls, NJ (US)

(72) Inventors: Lewis Scott AITKEN, Nottingham (GB); Lea Aurelie BOUCHE, Basel (CH); Wolfgang GUBA, Muellheim (DE); Georg JAESCHKE, Basel (CH); Stefanie Katharina MESCH, Basel (CH); Sandra STEINER, Sursee (CH); Andreas Michael TOSSTORFF, New York, NY (US)

(73) Assignee: Hoffmann-La Roche Inc., Little Falls, NJ (US)

(21) Appl. No.: 18/665,390

(22) Filed: May 15, 2024

Related U.S. Application Data
(63) Continuation of application No. PCT/EP2022/081866, filed on Nov. 15, 2022.

(30) Foreign Application Priority Data
Nov. 17, 2021 (EP) 21208773.8

(51) Int. Cl. C07D 495/04; A61K 31/5025; C07D 487/04; C07D 491/048; C07D 498/04

(52) U.S. Cl. CPC C07D 495/04; A61K 31/5025; C07D 487/04; C07D 491/048; C07D 498/04

(57) ABSTRACT

The invention relates to novel compounds having the general formula Ib

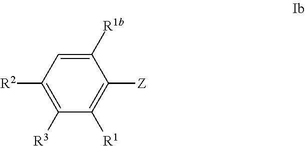

wherein R¹, R^{1b}, R², R³, and Z are as described herein, composition including the compounds and methods of using the compounds.

### CROSS-REFERENCE TO RELATED APPLICATIONS

This application is a Continuation of International Application No. PCT/EP2022/081866, filed Nov. 15, 2022, which claims benefit of priority to European Application No. 21208773.8 filed Nov. 17, 2021, the contents of which are incorporated herein by reference in its entirety.

### FIELD OF THE INVENTION

The present invention relates to organic compounds useful for therapy and/or prophylaxis in a mammal, and in particular to compounds that modulate NLRP3 inhibition.

The present invention provides novel compounds of formula Ib

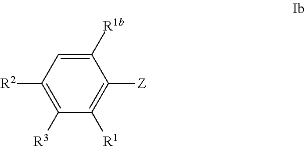

wherein
R¹ is H, haloalkyl or OH;
R^{1b} is H, halo or alkyl;
R² is halo, haloalkyl, haloalkoxy, nitrile or alkyl;
R³ is H;
or R² and R³, and the atoms to which they are attached, bond together to form either a 5-member heterocycle comprising 1 O heteroatom or a 4-member cycloalkyl ring;
Z is selected from ring-systems

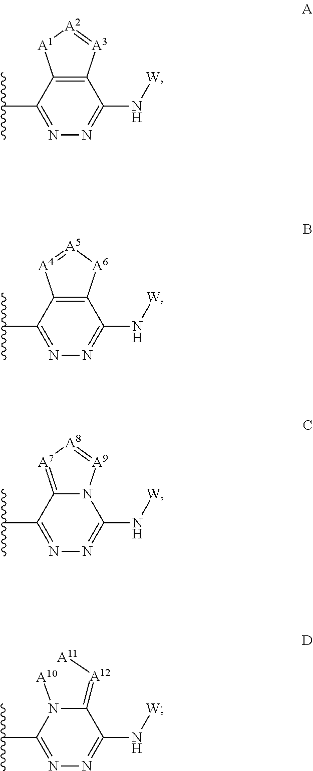

A¹ is S, NR^{X1} or O, wherein R^{X1} is H, alkyl, or cyclopropyl;
A² is CR^{Y1} or N, wherein R^{Y1} is H or alkyl;
A³ is CR^{Z1} or N, wherein R^{Z1} is H or alkyl;
wherein if A¹ is S or O then A² and A³ cannot both be N;
A⁴ is CR^{Z2} or N, wherein R^{Z2} is H or alkyl;
A⁵ is CR^{Y2} or N, wherein CR^{Y2} is H or alkyl;
A⁶ is S, NR^{X2} or O, wherein R^{X2} is H or alkyl;
wherein if A⁶ is S or O then A⁴ and A⁵ cannot both be N;
A⁷, A⁸ and A⁹ are independently CR^{W1} or N, wherein CR^{W1} is H or alkyl;
wherein A⁷, A⁸ and A⁹ cannot be all N;
A¹⁰, A¹¹ and A¹² are independently CR^{W2} or N, wherein CR^{W2} is H or alkyl;
wherein A¹⁰, A¹¹ and A¹² cannot be all N;
W is a substituted 4-member-cycloalkyl, a substituted 6-member-cycloalkyl, a substituted 6-member-heterocycle comprising a single heteroatom N, or 1,2,3,5,6,7,8,8a-octahydroindolizin-7-yl, wherein substituted 4-member-cycloalkyl is substituted with hydroxyl and methyl, substituted 6-member-cycloalkyl is substituted with OH, and substituted 6-member-heterocycle comprising a single heteroatom N is substituted with one or two substituents independently selected from alkyl, OH or halo; and pharmaceutically acceptable salts.

Furthermore, the invention includes all racemic mixtures, all their corresponding enantiomers and/or optical isomers.

### BACKGROUND OF THE INVENTION

The NOD-like receptor (NLR) family, pyrin domain-containing protein 3 (NLRP3) inflammasome is a component of the inflammatory process, and its aberrant activity is pathogenic in inherited disorders such as cryopyrin-associated periodic syndromes (CAPS) and complex diseases such as multiple sclerosis, type 2 diabetes, Alzheimer's disease and atherosclerosis.

NLRP3 is an intracellular signaling molecule that senses many pathogen-derived, environmental and host-derived factors. Upon activation, NLRP3 binds to apoptosis-associated speck-like protein containing a caspase activation and recruitment domain (ASC). ASC then polymerises to form a large aggregate known as an ASC speck. Polymerised ASC in turn interacts with the cysteine protease caspase-1 to form a complex termed the inflammasome. This results in the activation of caspase-1, which cleaves the precursor forms of the proinflammatory cytokines IL-1β and IL-18 (termed pro-IL-1β and pro-IL-18 respectively) to thereby activate these cytokines. Caspase-1 also mediates a type of inflammatory cell death known as pyroptosis. The ASC speck can also recruit and activate caspase-8, which can process pro-IL-1β and pro-IL-18 and trigger apoptotic cell death.

Caspase-1 cleaves pro-IL-1β and pro-IL-18 to their active forms, which are secreted from the cell. Active caspase-1 also cleaves gasdermin-D to trigger pyroptosis. Through its control of the pyroptotic cell death pathway, caspase-1 also mediates the release of alarmin molecules such as IL-33 and high mobility group box 1 protein (HMGB1). Caspase-1 also cleaves intracellular IL-1R2 resulting in its degradation and allowing the release of IL-1α. In human cells caspase-1 may also control the processing and secretion of IL-37. A number of other caspase-1 substrates such as components of the cytoskeleton and glycolysis pathway may contribute to caspase-1-dependent inflammation.

NLRP3-dependent ASC specks are released into the extracellular environment where they can activate caspase-1, induce processing of caspase-1 substrates and propagate inflammation.

Active cytokines derived from NLRP3 inflammasome activation are important drivers of inflammation and interact with other cytokine pathways to shape the immune response to infection and injury. For example, IL-1β signalling induces the secretion of the pro-inflammatory cytokines IL-6 and TNF. IL-1β and IL-18 synergise with IL-23 to induce IL-17 production by memory CD4 Th17 cells and by γδ T cells in the absence of T cell receptor engagement. IL-18 and IL-12 also synergise to induce IFN-γ production from memory T cells and NK cells driving a Th1 response.

The inherited CAPS diseases Muckle-Wells syndrome (MWS), familial cold autoinflammatory syndrome (FCAS) and neonatal-onset multisystem inflammatory disease (NOMID) are caused by gain-of-function mutations in NLRP3, thus defining NLRP3 as a critical component of the inflammatory process. NLRP3 has also been implicated in the pathogenesis of a number of complex diseases, notably including metabolic disorders such as type 2 diabetes, atherosclerosis, obesity and gout.

A role for NLRP3 in diseases of the central nervous system is emerging, and lung diseases have also been shown to be influenced by NLRP3. NLRP3 has also been suggested to have a role in a number of central nervous system conditions, including Parkinson's disease (PD), Alzheimer's disease (AD), dementia, Huntington's disease, cerebral malaria, brain injury from pneumococcal meningitis (Walsh et al., Nature Reviews, 15: 84-97, 2014, and Dempsey et al. Brain. Behav. Immun. 201761: 306-316). NLRP3 has also been shown to play a role in a number of lung diseases including chronic obstructive pulmonary disorder (COPD), asthma (including steroid-resistant asthma), asbestosis, and silicosis (De Nardo et al., Am. J. Pathol., 184: 42-54, 2014 and Kim et al. Am J Respir Crit Care Med. 2017 196(3): 283-97). Furthermore, NLRP3 has a role in the development of liver disease, kidney disease and aging. Many of these associations were defined using Nlrp3^{-/-} mice, but there have also been insights into the specific activation of NLRP3 in these diseases. In type 2 diabetes mellitus (T2D), the deposition of islet amyloid polypeptide in the pancreas activates NLRP3 and IL-1β signalling, resulting in cell death and inflammation.

Several small molecules have been shown to inhibit the NLRP3 inflammasome. Glyburide inhibits IL-1β production at micromolar concentrations in response to the activation of NLRP3 but not NLRC4 or NLRP1. Other previously characterised weak NLRP3 inhibitors include parthenolide, 3,4-methylenedioxy-β-nitrostyrene and dimethyl sulfoxide (DMSO), although these agents have limited potency and are nonspecific.

Current treatments for NLRP3-related diseases include biologic agents that target IL-1. These are the recombinant IL-1 receptor antagonist anakinra, the neutralizing IL-1β antibody canakinumab and the soluble decoy IL-1 receptor rilonacept. These approaches have proven successful in the treatment of CAPS, and these biologic agents have been used in clinical trials for other IL-1β-associated diseases.

There is a need to provide compounds with improved pharmacological and/or physiological and/or physicochemical properties and/or those that provide a useful alternative to known compounds.

### SUMMARY OF THE INVENTION

The present invention provides novel compounds of formula Ib

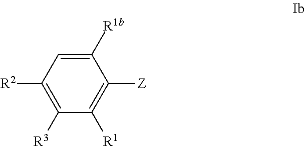

wherein
R¹ is H, haloalkyl or OH;
R^{1b} is H, halo or alkyl;
R² is halo, haloalkyl, haloalkoxy, nitrile or alkyl;
R³ is H;
or R² and R³, and the atoms to which they are attached, bond together to form either a 5-member heterocycle comprising 1 O heteroatom or a 4-member cycloalkyl ring;
Z is selected from ring-systems

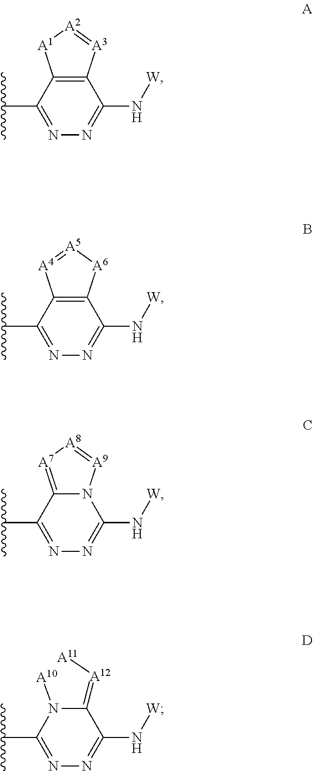

A¹ is S, NR^{X1} or O, wherein R^{X1} is H, alkyl, or cyclopropyl;
A² is CR^{Y1} or N, wherein R^{Y1} is H or alkyl;
A³ is CR^{Z1} or N, wherein R^{Z1} is H or alkyl;
wherein if A¹ is S or O then A² and A³ cannot both be N;
A⁴ is CR^{Z2} or N, wherein R^{Z2} is H or alkyl;
A⁵ is CR^{Y2} or N, wherein CR^{Y2} is H or alkyl;
A⁶ is S, NR^{X2} or O, wherein R^{X2} is H or alkyl;
wherein if A⁶ is S or O then A⁴ and A⁵ cannot both be N;
A⁷, A⁸ and A⁹ are independently CR^{W1} or N, wherein CR^{W1} is H or alkyl;
wherein A⁷, A⁸ and A⁹ cannot be all N;
A¹⁰, A¹¹ and A¹² are independently CR^{W2} or N, wherein CR^{W2} is H or alkyl; wherein A¹⁰, A¹¹ and A¹² cannot be all N;
W is a substituted 4-member-cycloalkyl, a substituted 6-member-cycloalkyl, a substituted 6-member-heterocycle comprising a single heteroatom N, or 1,2,3,5,6,7,8,8a-octahydroindolizin-7-yl, wherein substituted 4-member-cycloalkyl is substituted with hydroxyl and methyl, substituted 6-member-cycloalkyl is substituted with OH, and substituted 6-member-heterocycle comprising a single heteroatom N is substituted with one or two substituents independently selected from alkyl, OH or halo; and pharmaceutically acceptable salts.

### BRIEF DESCRIPTION OF DRAWINGS

FIG. 1 shows a voltage pattern used to stimulate cells to activate hERG channels and conduct outward IKhERG current, at a stimulation frequency of 0.1 Hz (6 bpm).

### DETAILED DESCRIPTION

The term “alkyl” denotes a monovalent linear or branched saturated hydrocarbon group of 1 to 6 carbon atoms. In some embodiments, if not otherwise described, alkyl comprises 1 to 6 carbon atoms (C₁₋₆-alkyl), or 1 to 4 carbon atoms (C₁₋₄-alkyl). Examples of C₁₋₆-alkyl include methyl, ethyl, propyl, isopropyl, n-butyl, iso-butyl, sec-butyl, tert-butyl and pentyl. Particular alkyl groups include methyl and ethyl.

The term “alkoxy” denotes a group of the formula —O—R′, wherein R′ is a C₁₋₆-alkyl group. Examples of C₁₋₆-alkoxy groups include methoxy, ethoxy, n-propoxy, isopropoxy, n-butoxy, isobutoxy and tert-butoxy.

The term “amino” denotes an —NH₂ group.

The term “cycloalkyl” denotes monocyclic or polycyclic saturated or partially unsaturated, non-aromatic hydrocarbon. In some embodiments, unless otherwise described, cycloalkyl comprises 3 to 8 carbon atoms, 3 to 6 carbon atoms, or 3 to 5 carbon atoms. In some embodiments, cycloalkyl is a saturated monocyclic or polycyclic hydrocarbon. Examples of cycloalkyl groups include cyclopropyl, cyclobutyl, cyclopentyl, cyclohexyl, octahydropentalenyl, spiro[3.3]heptanyl, and the like. Particular examples include cyclopropyl, cyclobutyl and cyclohexyl.

The term “halogen”, “halide” and “halo” are used interchangeably herein and denote fluoro, chloro, bromo or iodo. Particular halogens are fluoro and chloro. Preferred halogen is fluoro.

The term “haloalkyl” denotes a C₁₋₆-alkyl group wherein at least one of the hydrogen atoms of the C₁₋₆-alkyl group has been replaced by the same or different halogen atoms. Example of haloalkyl include fluoromethyl, difluoromethyl and trifluoromethyl. Particular example is trifluoromethyl.

The term “haloalkoxy” denotes a C₁₋₆-alkoxy group wherein at least one of the hydrogen atoms of the C₁₋₆-alkoxy group has been replaced by the same or different halogen atoms. Examples of haloalkoxy are difluoromethoxy, trifluoromethoxy, difluoroethoxy and trifluoroethoxy. Particular example is trifluromethoxy.

The term “heterocycle ring” denotes a monovalent saturated or partly unsaturated mono- or bicyclic ring system of 4 to 9 ring atoms, comprising 1, 2, or 3 ring heteroatoms selected from N, O and S, the remaining ring atoms being carbon. Examples for monocyclic saturated heterocycle rings are azetidinyl, diazepanyl, pyrrolidinyl, tetrahydrofuranyl, pyrazolidinyl, imidazolidinyl, oxazolidinyl, isoxazolidinyl, thiazolidinyl, piperidinyl, tetrahydropyranyl, tetrahydrothiopyranyl, morpholinyl, and piperazinyl. Examples of polycyclic saturated heterocycle rings are azaspiroheptanyl, diazaspiroheptanyl. azaspirooctanyl, diazospirooctanyl, diazaspirononanyl, oxaazaspirooctanyl, and oxadiazaspirononanyl. Particular example of heterocycle rings are azetidinyl, pyrrolidinyl, piperidinyl, morpholinyl, tetrahydropyranyl and piperazinyl. More particular examples of heterocycle rings are pyrrolidinyl, piperidinyl, morpholinyl, and piperazinyl. Preferred example of a heterocycle ring is piperidinyl. Another preferred example of a heterocycle ring is an oxolanyl ring.

The term “hydroxy” denotes a —OH group.

The term “nitrile” denotes a —C≡N group.

The term “pharmaceutically acceptable salts” refers to those salts which retain the biological effectiveness and properties of the free bases or free acids, which are not biologically or otherwise undesirable. The salts are formed with inorganic acids such as trifluoroacetic acid, hydrochloric acid, hydrobromic acid, sulfuric acid, nitric acid, phosphoric acid, particularly hydrochloric acid, and organic acids such as formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein. In addition these salts may be prepared from addition of an inorganic base or an organic base to the free acid. Salts derived from an inorganic base include, but are not limited to, the sodium, potassium, lithium, ammonium, calcium, magnesium salts. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyamine resins. The compound of formula I can also be present in the form of zwitterions. Particularly preferred pharmaceutically acceptable salts of compounds of formula I are the salts formed with formic acid and the salts formed with hydrochloric acid yielding a hydrochloride, dihydrochloride or trihydrochloride salt.

The abbreviation uM means microMolar and is equivalent to the symbol μM.

The abbreviation uL means microliter and is equivalent to the symbol μL.

The abbreviation ug means microgram and is equivalent to the symbol μg.

The compounds of formula Ib can contain several asymmetric centers and can be present in the form of optically pure enantiomers, mixtures of enantiomers such as, for example, racemates, optically pure diastereoisomers, mixtures of diastereoisomers, diastereoisomeric racemates or mixtures of diastereoisomeric racemates.

The compounds of formula I can contain several asymmetric centers and can be present in the form of optically pure enantiomers, mixtures of enantiomers such as, for example, racemates, optically pure diastereoisomers, mixtures of diastereoisomers, diastereoisomeric racemates or mixtures of diastereoisomeric racemates.

According to the Cahn-Ingold-Prelog Convention the asymmetric carbon atom can be of the “R” or “S” configuration.

Also an embodiment of the present invention provides compounds according to formula Ib as described herein and pharmaceutically acceptable salts or esters thereof, in particular compounds according to formula I as described herein and pharmaceutically acceptable salts thereof, more particularly compounds according to formula Ib as described herein.

Also an embodiment of the present invention provides compounds according to formula I as described herein and pharmaceutically acceptable salts or esters thereof, in particular compounds according to formula I as described herein and pharmaceutically acceptable salts thereof, more particularly compounds according to formula I as described herein.

An embodiment of the present invention provides compounds according to formula Ib as described herein, wherein Z is selected from
Ring System A, wherein
   A¹ is S, NR^{X1} or O, wherein R^{X1} is H or alkyl;
   A² is CR^{Y1} or N, wherein R^{Y1} is H;
   A³ is CR^{Z1} or N, wherein R^{Z1} is H or alkyl;
   wherein if A¹ is S or O then neither A² nor A³ can be N; or
Ring System B, wherein
   A⁴ is CR^{Z2}, wherein R^{Z2} is H;
   A⁵ is CR^{Y2} or N, wherein CR^{Y2} is H;
   A⁶ is S or NR^{X2}, wherein R^{X2} is alkyl;
   wherein if A⁶ is S then neither A⁴ nor A⁵ can be N.

An embodiment of the present invention provides compounds according to formula Ib as described herein, wherein
A¹ is S, NR^{X1} or O, wherein R^{X1} is H or alkyl;
A² is CR^{Y1} or N, wherein R^{Y1} is H;
A³ is CR^{Z1} or N, wherein R^{Z1} is H or alkyl;
wherein if A¹ is S or O then neither A² nor A³ can be N.

An embodiment of the present invention provides compounds according to formula Ib as described herein, wherein Z is Ring System A, wherein Ring System A comprises 2 N heteroatoms.

An embodiment of the present invention provides compounds according to formula Ib as described herein, wherein Z is Ring System A, wherein
A¹ is NR^{X1}, wherein R^{X1} is alkyl;
A² is N; and
A³ is CR^{Z1}, wherein R^{Z1} is H.

An embodiment of the present invention provides compounds according to formula Ib as described herein, wherein R¹ is H or OH.

An embodiment of the present invention provides compounds according to formula Ib as described herein, wherein R¹ is OH.

An embodiment of the present invention provides compounds according to formula Ib as described herein, wherein R^{1b} is H.

An embodiment of the present invention provides compounds according to formula Ib as described herein, wherein R² is halo, haloalkyl, or haloalkoxy and R³ is H; or
R² and R³, and the atoms to which they are attached, bond together to form either a heterocycle comprising 1 O heteroatom or a cycloalkyl ring.

An embodiment of the present invention provides compounds according to formula Ib as described herein, wherein R² and R³, and the atoms to which they are attached, bond together to form either a heterocycle comprising 1 O heteroatom or a cycloalkyl ring.

An embodiment of the present invention provides compounds according to formula Ib as described herein, wherein R² and R³, and the atoms to which they are attached, bond together to form either a 5-member heterocycle comprising 1 O heteroatom or a 4-member cycloalkyl ring.

An embodiment of the present invention provides compounds according to formula Ib as described herein, wherein R² and R³, and the atoms to which they are attached, bond together to form a cycloalkyl ring.

An embodiment of the present invention provides compounds according to formula Ib as described herein, wherein R² and R³, and the atoms to which they are attached, bond together to form a 4-member cycloalkyl ring.

An embodiment of the present invention provides compounds according to formula Ib as described herein, wherein R² and R³, and the atoms to which they are attached, bond together to form either a heterocycle comprising 1 O heteroatom or a cycloalkyl ring, and Z is Ring System A, wherein Ring System A comprises 2 N heteroatoms.

An embodiment of the present invention provides compounds according to formula Ib as described herein, wherein R² and R³, and the atoms to which they are attached, bond together to form a cycloalkyl ring, and Z is Ring System A, wherein Ring System A comprises 2 N heteroatoms.

An embodiment of the present invention provides compounds according to formula Ib as described herein, wherein W is a substituted 4-member-cycloalkyl, a substituted 6-member-cycloalkyl, or a substituted 6-member-heterocycle comprising a single heteroatom N, wherein substituted 4-member-cycloalkyl is substituted with hydroxyl and methyl, substituted 6-member-cycloalkyl is substituted with OH, and substituted 6-member-heterocycle comprising a single heteroatom N is substituted with one or two substituents independently selected from alkyl and OH.

An embodiment of the present invention provides compounds according to formula Ib as described herein, wherein W is ethylpiperidyl or 1-ethyl-piperidin-3-ol.

An embodiment of the present invention provides compounds according to formula Ib as described herein, wherein W is ethylpiperidyl.

An embodiment of the present invention provides compounds according to formula Ib as described herein, wherein
R¹ is H or OH;
R^{1b} is H, halo or alkyl;
R² is halo, haloalkyl or haloalkoxy;
R³ is H;
or R² and R³, and the atoms to which they are attached, bond together to form either a 5-member heterocycle comprising 1 O heteroatom or a 4-member cycloalkyl ring;
Z is selected from
   Ring System A, wherein
      A¹ is S, NR^{X1} or O, wherein R^{X1} is H or alkyl;
      A² is CR^{Y1} or N, wherein R^{Y1} is H;
      A³ is CR^{Z1} or N, wherein R^{Z1} is H or alkyl;
      wherein if A¹ is S or O then neither A² nor A³ can be N; or
   Ring System B, wherein
      A⁴ is CR^{Z2}, wherein R^{Z2} is H;
      A⁵ is CR^{Y2} or N, wherein CR^{Y2} is H;
      A⁶ is S or NR^{X2}, wherein R^{X2} is alkyl;
      wherein if A⁶ is S then neither A⁴ nor A⁵ can be N;
W is a substituted 4-member-cycloalkyl, a substituted 6-member-cycloalkyl, or a substituted 6-member-heterocycle comprising a single heteroatom N, wherein substituted 4-member-cycloalkyl is substituted with hydroxyl and methyl, substituted 6-member-cycloalkyl is substituted with OH, and substituted 6-member-heterocycle comprising a single heteroatom N is substituted with one or two substituents independently selected from alkyl and OH;
and pharmaceutical acceptable salts thereof.

An embodiment of the present invention provides compounds according to formula Ib as described herein, wherein
R¹ is H or OH;
R^{1b} is H, halo or alkyl;
R² is halo, haloalkyl or haloalkoxy;
R³ is H;
or R² and R³, and the atoms to which they are attached, bond together to form either a 5-member heterocycle comprising 1 O heteroatom or a 4-member cycloalkyl ring;
Z is Ring System A, wherein
   A¹ is S, NR^{X1} or O, wherein R^{X1} is H or alkyl;
   A² is CR^{Y1} or N, wherein R^{Y1} is H;
   A³ is CR^{Z1} or N, wherein R^{Z1} is H or alkyl;
   wherein if A¹ is S or O then neither A² nor A³ can be N;
W is a substituted 4-member-cycloalkyl, a substituted 6-member-cycloalkyl, or a substituted 6-member-heterocycle comprising a single heteroatom N, wherein substituted 4-member-cycloalkyl is substituted with hydroxyl and methyl, substituted 6-member-cycloalkyl is substituted with OH, and substituted 6-member-heterocycle comprising a single heteroatom N is substituted with one or two substituents independently selected from alkyl and OH;
and pharmaceutical acceptable salts thereof.

An embodiment of the present invention provides compounds according to formula Ib as described herein, wherein
R¹ is OH;
R^{1b} is H;
R² and R³, and the atoms to which they are attached, bond together to form either a 5-member heterocycle comprising 1 O heteroatom or a 4-member cycloalkyl ring;
Z is Ring System A, wherein
   A¹ is NR^{X1}, wherein R^{X1} is alkyl;
   A² is N;
   A³ is CR^{Z1}, wherein R^{Z1} is H;
W is ethylpiperidyl or 1-ethyl-piperidin-3-ol;
and pharmaceutical acceptable salts thereof.

An embodiment of the present invention provides compounds according to formula Ib as described herein, wherein
R¹ is OH;
R^{1b} is H;
R² and R³, and the atoms to which they are attached, bond together to form either a 4-member cycloalkyl ring;
Z is Ring System A, wherein
   A¹ is NR^{X1}, wherein R^{X1} is alkyl;
   A² is N;
   A³ is CR^{Z1}, wherein R^{Z1} is H;
W is ethylpiperidyl;
and pharmaceutical acceptable salts thereof.

Particular examples of compounds of formula Ib as described herein are selected from
(rac)-2-[7-[(1-Ethyl-3-piperidyl)amino]thieno[2,3-d]pyridazin-4-yl]-5(trifluoromethyl)phenol; formic acid;
(rac)-2-[7-[(1-Ethyl-3-piperidyl)amino]thieno[2,3-d]pyridazin-4-yl]-5(trifluoromethyl)phenol;
(rac)-2-[4-[(1-Ethyl-3-piperidyl)amino]thieno[2,3-d]pyridazin-7-yl]-5-(trifluoromethyl)phenol; formic acid;
(rac)-2-[4-[(1-Ethyl-3-piperidyl)amino]thieno[2,3-d]pyridazin-7-yl]-5-(trifluoromethyl)phenol;
(rac)-N-(1-ethyl-3-piperidyl)-4-[4-(trifluoromethyl)phenyl]thieno[2,3-d]pyridazin-7-amine;
(rac)-N-(1-ethyl-3-piperidyl)-7-[4-(trifluoromethyl)phenyl]thieno[2,3-d]pyridazin-4-amine;
2-[4-[[(3R)-1-Ethyl-3-piperidyl]amino]-1-methyl-pyrrolo[2,3-d]pyridazin-7-yl]-5-(trifluoromethyl)phenol; hydrochloride;
2-[4-[[(3R)-1-ethyl-3-piperidyl]amino]-1-methyl-pyrrolo[2,3-d]pyridazin-7-yl]-5-(trifluoromethyl)phenol; 2,2,2-trifluoroacetic acid;
2-[4-[[(3R)-1-ethyl-3-piperidyl]amino]-1-methyl-pyrrolo[2,3-d]pyridazin-7-yl]-5-(trifluoromethyl)phenol;
2-[4-[[(3R)-1-Ethyl-3-piperidyl]amino]-1-methyl-pyrazolo[3,4-d]pyridazin-7-yl]-5-(trifluoromethyl)phenol; 2,2,2-trifluoroacetic acid;
2-[4-[[(3R)-1-Ethyl-3-piperidyl]amino]-1-methyl-pyrazolo[3,4-d]pyridazin-7-yl]-5-(trifluoromethyl)phenol; and
2-[4-[[(3R)-1-ethyl-3-piperidyl]amino]furo[2,3-d]pyridazin-7-yl]-5-(trifluoromethyl)phenol;
and pharmaceutically acceptable salts thereof.

Also particular examples of compounds of formula Ib as described herein are selected from
2-[4-[[(3R)-1-Ethyl-3-piperidyl]amino]-1-methyl-imidazo[4,5-d]pyridazin-7-yl]-5-(trifluoromethyl)phenol;
2-[7-[[(3R)-1-Ethyl-3-piperidyl]amino]-1-methyl-pyrazolo[3,4-d]pyridazin-4-yl]-5-(trifluoromethyl)phenol;
2-[4-[[(3R)-1-Ethyl-3-piperidyl]amino]-1-methyl-pyrazolo[3,4-d]pyridazin-7-yl]-5-(trifluoromethoxy)phenol;
2-[4-[[(1R,2R)-2-Hydroxycyclohexyl]amino]-1-methyl-pyrazolo[3,4-d]pyridazin-7-yl]-5-(trifluoromethyl)phenol;
5-Chloro-2-[1-methyl-4-[[(3R)-1-ethyl-3-piperidyl]amino]pyrazolo[3,4-d]pyridazin-7-yl]phenol;
2-[4-[[(3R)-1-Ethyl-3-piperidyl]amino]-3-methyl-isoxazolo[4,5-d]pyridazin-7-yl]-5-(trifluoromethyl)phenol;
2-[4-[[(3R)-1-Ethyl-3-piperidyl]amino]-1H-pyrazolo[3,4-d]pyridazin-7-yl]-3-methyl-5-(trifluoromethyl)phenol;
2-[4-[[(3R)-1-Ethyl-3-piperidyl]amino]-1-methyl-pyrazolo[3,4-d]pyridazin-7-yl]-3-fluoro-5-(trifluoromethyl)phenol;
2-[4-[(3-Hydroxy-3-methyl-cyclobutyl)amino]-1-methyl-pyrazolo[3,4-d]pyridazin-7-yl]-5-(trifluoromethyl)phenol;
5-[4-[[(3R)-1-Ethyl-3-piperidyl]amino]-1-methyl-pyrazolo[3,4-d]pyridazin-7-yl]-2,3-dihydrobenzofuran-4-ol;
3-[4-[[(3R)-1-Ethyl-3-piperidyl]amino]-1-methyl-pyrazolo[3,4-d]pyridazin-7-yl]bicyclo[4.2.0]octa-1(6),2,4-trien-2-ol;
(3S,5R)-1-Ethyl-5-[[7-(4-hydroxy-2,3-dihydrobenzofuran-5-yl)-1-methyl-pyrazolo[3,4-d]pyridazin-4-yl]amino]piperidin-3-ol;
and pharmaceutically acceptable salts thereof.

Preferred examples of compounds of formula Ib as described herein are selected from
5-[4-[[(3R)-1-Ethyl-3-piperidyl]amino]-1-methyl-pyrazolo[3,4-d]pyridazin-7-yl]-2,3-dihydrobenzofuran-4-ol;
3-[4-[[(3R)-1-Ethyl-3-piperidyl]amino]-1-methyl-pyrazolo[3,4-d]pyridazin-7-yl]bicyclo[4.2.0]octa-1(6),2,4-trien-2-ol;
(3S,5R)-1-Ethyl-5-[[7-(4-hydroxy-2,3-dihydrobenzofuran-5-yl)-1-methyl-pyrazolo[3,4-d]pyridazin-4-yl]amino]piperidin-3-ol;
and pharmaceutically acceptable salts thereof.

Most preferred examples of compounds of formula Ib as described herein is 3-[4-[[(3R)-1-ethyl-3-piperidyl]amino]-1-methyl-pyrazolo[3,4-d]pyridazin-7-yl]bicyclo[4.2.0]octa-1(6),2,4-trien-2-ol, and pharmaceutically acceptable salts thereof.

An embodiment of the present invention provides compounds of formula I, wherein the compound of formula I is a compound of formula Ib,

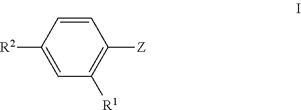

wherein
R¹ is H, haloalkyl or OH;
R² is halo, haloalkyl, haloalkoxy, nitrile or alkyl;
Z is selected from ring-systems

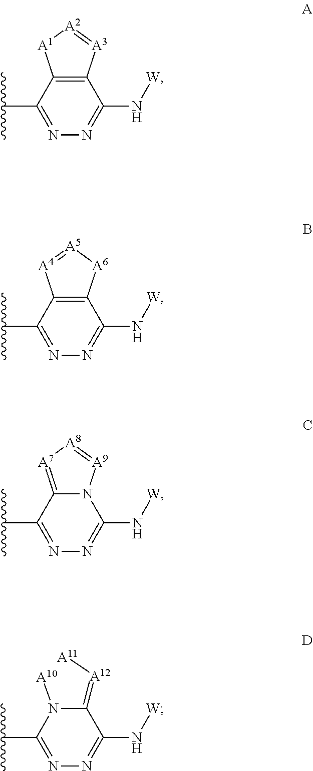

A¹ is S, NR^{X1} or O, wherein R^{X1} is H, alkyl, or cyclopropyl;
A² is CR^{Y1} or N, wherein R^{Y1} is H or alkyl;
A³ is CR^{Z1} or N, wherein R^{Z1} is H or alkyl;
wherein if A¹ is S or O then A² and A³ cannot both be N;
A⁴ is CR^{Z2} or N, wherein R^{Z2} is H or alkyl;
A⁵ is CR^{Y2} or N, wherein CR^{Y2} is H or alkyl;
A⁶ is S, NR^{X2} or O, wherein R^{X2} is H or alkyl;
wherein if A⁶ is S or O then A⁴ and A⁵ cannot both be N;
A⁷, A⁸ and A⁹ are independently CR^{W1} or N, wherein CR^{W1} is H or alkyl;
wherein A⁷, A⁸ and A⁹ cannot be all N;
A¹⁰, A¹¹ and A¹² are independently CR^{W2} or N, wherein CR^{W2} is H or alkyl;
wherein A¹⁰, A¹¹ and A¹² cannot be all N;
W is a substituted 4-member-cycloalkyl, a substituted 6-member-cycloalkyl, a substituted 6-member-heterocycle comprising a single heteroatom N, or 1,2,3,5,6,7,8,8a-octahydroindolizin-7-yl, wherein substituted 4-member-cycloalkyl is substituted with hydroxyl and methyl, substituted 6-member-cycloalkyl is substituted with OH, and substituted 6-member-heterocycle comprising a single heteroatom N is substituted with alkyl, OH or halo;
and pharmaceutically acceptable salts.

An embodiment of the present invention provides compounds according to formula I as described herein, wherein R¹ is H or OH.

An embodiment of the present invention provides compounds according to formula I as described herein, wherein R¹ is OH.

An embodiment of the present invention provides compounds according to formula I as described herein, wherein R² is halo, haloalkyl, or haloalkoxy.

An embodiment of the present invention provides compounds according to formula I as described herein, wherein R² is halo or haloalkyl.

An embodiment of the present invention provides compounds according to formula I as described herein, wherein R² is haloalkyl.

An embodiment of the present invention provides compounds according to formula I as described herein, wherein Z is selected from
Ring System A, wherein
   A¹ is S, NR^{X1} or O, wherein R^{X1} is alkyl;
   A² is CR^{Y1} or N, wherein R^{Y1} is H;
   A³ is CR^{Z1} or N, wherein R^{Z1} is H;
   wherein if A¹ is S or O then neither A² nor A³ can be N;
Ring System B, wherein
   A⁴ is CR^{Z2} or N, wherein R^{Z2} is H;
   A⁵ is CR^{Y2} or N, wherein CR^{Y2} is H;
   A⁶ is S, NR^{X2} or O, wherein R^{X2} is alkyl;
   wherein if A⁶ is S or O then neither A⁴ nor A⁵ can be N;
Ring System C, wherein A⁷ is N and A⁸ and A⁹ are both CH;
Ring System D, wherein A¹⁰ and A¹¹ are both CH and A¹² is N;
and W is 1-ethyl-3-piperidyl or 1-methyl-3-piperidyl.

An embodiment of the present invention provides compounds according to formula I as described herein, wherein Z is is Ring System A, wherein
A¹ is NR^{X1}, wherein R^{X1} is alkyl;
A² is CR^{Y1} or N, wherein R^{Y1} is H;
A³ is CR^{Z1}, wherein R^{Z1} is H;
and W is 1-ethyl-3-piperidyl or 1-methyl-3-piperidyl.

An embodiment of the present invention provides compounds according to formula I as described herein, wherein Z is is Ring System A, wherein
A¹ is NR^{X1}, wherein R^{X1} is methyl;
A² is CR^{Y1} or N, wherein R^{Y1} is H;
A³ is CR^{Z1}, wherein R^{Z1} is H;
and W is 1-ethyl-3-piperidyl.

An embodiment of the present invention provides compounds according to formula I as described herein, wherein Z is is Ring System A, wherein
A¹ is NR^{X1}, wherein R^{X1} is methyl;
A² is N;
A³ is CR^{Z1}, wherein R^{Z1} is H;
and W is 1-ethyl-3-piperidyl.

An embodiment of the present invention provides compounds according to formula I as described here, wherein
R¹ is H or OH;
R² is halo, haloalkyl or haloalkoxy;
Z is selected from
   Ring System A, wherein
      A¹ is S, NR^{X1} or O, wherein R^{X1} is alkyl;
      A² is CR^{Y1} or N, wherein R^{Y1} is H;
      A³ is CR^{Z1} or N, wherein R^{Z1} is H;
      wherein if A¹ is S or O then neither A² nor A³ can be N;
   Ring System B, wherein
      A⁴ is CR^{Z2} or N, wherein R^{Z2} is H;
      A⁵ is CR^{Y2} or N, wherein CR^{Y2} is H;
      A⁶ is S, NR^{X2} or O, wherein R^{X2} is alkyl;
      wherein if A⁶ is S or O then neither A⁴ nor A⁵ can be N;
   Ring System C, wherein A⁷ is N and both A⁸ and A⁹ are CH;
   Ring System D, wherein A¹⁰ and A¹¹ are CH and A¹² is N;
   and W is 1-ethyl-3-piperidyl or 1-methyl-3-piperidyl;
and pharmaceutical acceptable salts thereof.

An embodiment of the present invention provides compounds according to formula I as described here, wherein
R¹ is H or OH;
R² is haloalkyl;
Z is selected from Ring System A, wherein
   A¹ is NR^{X1}, wherein R^{X1} is alkyl;
   A² is CR^{Y1} or N, wherein R^{Y1} is H;
   A³ is CR^{Z1}, wherein R^{Z1} is H;
   and W is 1-ethyl-3-piperidyl or 1-methyl-3-piperidyl;
and pharmaceutical acceptable salts thereof.

An embodiment of the present invention provides compounds according to formula I as described here, wherein
R¹ is H or OH;
R² is haloalkyl;
Z is selected from Ring System A, wherein
   A¹ is NR^{X1}, wherein R^{X1} is methyl;
   A² is CR^{Y1} or N, wherein R^{Y1} is H;
   A³ is CR^{Z1}, wherein R^{Z1} is H;
   and W is 1-ethyl-3-piperidyl;
and pharmaceutical acceptable salts thereof.

An embodiment of the present invention provides compounds according to formula I as described here, wherein
R¹ is H or OH;
R² is haloalkyl;
Z is selected from Ring System A, wherein
   A¹ is NR^{X1}, wherein R^{X1} is methyl;
   A² is N;
   A³ is CR^{Z1}, wherein R^{Z1} is H;
   and W is 1-ethyl-3-piperidyl;
and pharmaceutical acceptable salts thereof.

An embodiment of the present invention provides compounds according to formula I as described here, wherein
R¹ is OH;
R² is haloalkyl;
Z is selected from Ring System A, wherein
   A¹ is NR^{X1}, wherein R^{X1} is methyl;
   A² is N;
   A³ is CR^{Z1}, wherein R^{Z1} is H;
   and W is 1-ethyl-3-piperidyl;
and pharmaceutical acceptable salts thereof.

Particular examples of compounds of formula I as described herein are selected from
(rac)-2-[7-[(1-Ethyl-3-piperidyl)amino]thieno[2,3-d]pyridazin-4-yl]-5(trifluoromethyl)phenol; formic acid;
(rac)-2-[7-[(1-Ethyl-3-piperidyl)amino]thieno[2,3-d]pyridazin-4-yl]-5(trifluoromethyl)phenol;
(rac)-2-[4-[(1-Ethyl-3-piperidyl)amino]thieno[2,3-d]pyridazin-7-yl]-5-(trifluoromethyl)phenol; formic acid;
(rac)-2-[4-[(1-Ethyl-3-piperidyl)amino]thieno[2,3-d]pyridazin-7-yl]-5-(trifluoromethyl)phenol;
(rac)-N-(1-ethyl-3-piperidyl)-4-[4-(trifluoromethyl)phenyl]thieno[2,3-d]pyridazin-7-amine;
(rac)-N-(1-ethyl-3-piperidyl)-7-[4-(trifluoromethyl)phenyl]thieno[2,3-d]pyridazin-4-amine;
2-[4-[[(3R)-1-Ethyl-3-piperidyl]amino]-1-methyl-pyrrolo[2,3-d]pyridazin-7-yl]-5-(trifluoromethyl)phenol; hydrochloride;
2-[4-[[(3R)-1-ethyl-3-piperidyl]amino]-1-methyl-pyrrolo[2,3-d]pyridazin-7-yl]-5-(trifluoromethyl)phenol; 2,2,2-trifluoroacetic acid;
2-[4-[[(3R)-1-ethyl-3-piperidyl]amino]-1-methyl-pyrrolo[2,3-d]pyridazin-7-yl]-5-(trifluoromethyl)phenol;
2-[4-[[(3R)-1-Ethyl-3-piperidyl]amino]-1-methyl-pyrazolo[3,4-d]pyridazin-7-yl]-5-(trifluoromethyl)phenol; 2,2,2-trifluoroacetic acid;
2-[4-[[(3R)-1-Ethyl-3-piperidyl]amino]-1-methyl-pyrazolo[3,4-d]pyridazin-7-yl]-5-(trifluoromethyl)phenol; and
2-[4-[[(3R)-1-ethyl-3-piperidyl]amino]furo[2,3-d]pyridazin-7-yl]-5-(trifluoromethyl)phenol;
and pharmaceutically acceptable salts thereof.

Preferred examples of compounds of formula I as described herein are selected from
(rac)-2-[7-[(1-Ethyl-3-piperidyl)amino]thieno[2,3-d]pyridazin-4-yl]-5(trifluoromethyl)phenol; formic acid;
(rac)-2-[7-[(1-Ethyl-3-piperidyl)amino]thieno[2,3-d]pyridazin-4-yl]-5(trifluoromethyl)phenol;
(rac)-N-(1-ethyl-3-piperidyl)-4-[4-(trifluoromethyl)phenyl]thieno[2,3-d]pyridazin-7-amine;
2-[4-[[(3R)-1-ethyl-3-piperidyl]amino]-1-methyl-pyrrolo[2,3-d]pyridazin-7-yl]-5-(trifluoromethyl)phenol; 2,2,2-trifluoroacetic acid;
2-[4-[[(3R)-1-Ethyl-3-piperidyl]amino]-1-methyl-pyrrolo[2,3-d]pyridazin-7-yl]-5-(trifluoromethyl)phenol; hydrochloride;
2-[4-[[(3R)-1-Ethyl-3-piperidyl]amino]-1-methyl-pyrrolo[2,3-d]pyridazin-7-yl]-5-(trifluoromethyl)phenol;
2-[4-[[(3R)-1-Ethyl-3-piperidyl]amino]-1-methyl-pyrazolo[3,4-d]pyridazin-7-yl]-5-(trifluoromethyl)phenol; 2,2,2-trifluoroacetic acid;
2-[4-[[(3R)-1-Ethyl-3-piperidyl]amino]-1-methyl-pyrazolo[3,4-d]pyridazin-7-yl]-5-(trifluoromethyl)phenol; and
2-[4-[[(3R)-1-ethyl-3-piperidyl]amino]furo[2,3-d]pyridazin-7-yl]-5-(trifluoromethyl)phenol;
and pharmaceutically acceptable salts thereof.

More preferred example of compounds of formula I as described herein are
2-[4-[[(3R)-1-Ethyl-3-piperidyl]amino]-1-methyl-pyrazolo[3,4-d]pyridazin-7-yl]-5-(trifluoromethyl)phenol; 2,2,2-trifluoroacetic acid or
2-[4-[[(3R)-1-Ethyl-3-piperidyl]amino]-1-methyl-pyrazolo[3,4-d]pyridazin-7-yl]-5-(trifluoromethyl)phenol;
and pharmaceutically acceptable salts thereof.

Most preferred example of compounds of formula I as described herein is
2-[4-[[(3R)-1-Ethyl-3-piperidyl]amino]-1-methyl-pyrazolo[3,4-d]pyridazin-7-yl]-5-(trifluoromethyl)phenol; 2,2,2-trifluoroacetic acid or
and pharmaceutically acceptable salts thereof

Another embodiment of the invention provides a pharmaceutical composition or medicament containing a compound of the invention and a therapeutically inert carrier, diluent or excipient, as well as a method of using the compounds of the invention to prepare such composition and medicament. In one example, the compound of formula I may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula I is formulated in an acetate buffer, at pH 5. In another embodiment, the compound of formula I is sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

The compounds of formula I and their pharmaceutically acceptable salts can be processed with pharmaceutically inert, inorganic or organic adjuvants for the production of tablets, coated tablets, dragées, hard gelatin capsules, injection solutions or topical formulations Lactose, corn starch or derivatives thereof, talc, stearic acid or its salts etc. can be used, for example, as such adjuvants for tablets, dragées and hard gelatin capsules.

Suitable adjuvants for soft gelatin capsules, are, for example, vegetable oils, waxes, fats, semi-solid substances and liquid polyols, etc.

Suitable adjuvants for the production of solutions and syrups are, for example, water, polyols, saccharose, invert sugar, glucose, etc.

Suitable adjuvants for injection solutions are, for example, water, alcohols, polyols, glycerol, vegetable oils, etc.

Suitable adjuvants for suppositories are, for example, natural or hardened oils, waxes, fats, semi-solid or liquid polyols, etc.

Suitable adjuvants for topical ocular formulations are, for example, cyclodextrins, mannitol or many other carriers and excipients known in the art.

Moreover, the pharmaceutical preparations can contain preservatives, solubilizers, viscosity-increasing substances, stabilizers, wetting agents, emulsifiers, sweeteners, colorants, flavorants, salts for varying the osmotic pressure, buffers, masking agents or antioxidants. They can also contain still other therapeutically valuable substances.

The dosage can vary in wide limits and will, of course, be fitted to the individual requirements in each particular case. In general, in the case of oral administration a daily dosage of about 0.1 mg to 20 mg per kg body weight, preferably about 0.5 mg to 4 mg per kg body weight (e.g. about 300 mg per person), divided into preferably 1-3 individual doses, which can consist, for example, of the same amounts, should it be appropriate. In the case of topical administration, the formulation can contain 0.001% to 15% by weight of medicament and the required dose, which can be between 0.1 and 25 mg in can be administered either by single dose per day or per week, or by multiple doses (2 to 4) per day, or by multiple doses per week It will, however, be clear that the upper or lower limit given herein can be exceeded when this is shown to be indicated.

An embodiment of the present invention is a compound according to formula Ib as described herein for use as a therapeutically active substance.

An embodiment of the present invention is a compound according to formula Ib as described herein for use in the treatment or prevention of a disease, disorder or condition, wherein the disease, disorder or condition is responsive to NLRP3 inhibition.

An embodiment of the present invention is a compound according to formula Ib as described herein for the treatment or prophylaxis of a disease, disorder or condition, wherein the disorder or condition is responsive to NLRP3 inhibition.

An embodiment of the present invention is a compound according to formula I as described herein for use as a therapeutically active substance.

An embodiment of the present invention is a compound according to formula I as described herein for use in the treatment or prevention of a disease, disorder or condition, wherein the disease, disorder or condition is responsive to NLRP3 inhibition.

An embodiment of the present invention is a compound according to formula I as described herein for the treatment or prophylaxis of a disease, disorder or condition, wherein the disorder or condition is responsive to NLRP3 inhibition.

As used herein, the term “NLRP3 inhibition” refers to the complete or partial reduction in the level of activity of NLRP3 and includes, for example, the inhibition of active NLRP3 and/or the inhibition of activation of NLRP3.

There is evidence for a role of NLRP3-induced IL-1 and IL-18 in the inflammatory responses occurring in connection with, or as a result of, a multitude of different disorders (Menu et al., Clinical and Experimental Immunology, 166: 1-15, 2011; Strowig et al., Nature, 481: 278-286, 2012).

In one embodiment, the disease, disorder or condition is selected from:
(i) inflammation;
(ii) an auto-immune disease;
(iii) cancer;
(iv) an infection;
(v) a central nervous system disease;
(vi) a metabolic disease;
(vii) a cardiovascular disease;
(viii) a respiratory disease;
(ix) a liver disease;
(x) a renal disease;
(xi) an ocular disease;
(xii) a skin disease;
(xiii) a lymphatic condition;
(xiv) a psychological disorder;
(xv) graft versus host disease;
(xvi) allodynia;
(xvii) a condition associated with diabetes; and
(xviii) any disease where an individual has been determined to carry a germline or somatic non-silent mutation in NLRP3

In another embodiment, the disease, disorder or condition is selected from:
(i) cancer;
(ii) an infection;
(iii) a central nervous system disease;
(iv) a cardiovascular disease;
(v) a liver disease;
(vi) an ocular disease; or
(vii) a skin disease.

In a further typical embodiment of the invention, the disease, disorder or condition is inflammation. Examples of inflammation that may be treated or prevented include inflammatory responses occurring in connection with, or as a result of:
(i) a skin condition such as contact hypersensitivity, bullous pemphigoid, sunburn, psoriasis, atopical dermatitis, contact dermatitis, allergic contact dermatitis, seborrhoetic dermatitis, lichen planus, scleroderma, pemphigus, epidermolysis bullosa, urticaria, erythemas, or alopecia;
(ii) a joint condition such as osteoarthritis, systemic juvenile idiopathic arthritis, adult-onset Still's disease, relapsing polychondritis, rheumatoid arthritis, juvenile chronic arthritis, gout, or a seronegative spondyloarthropathy (e.g. ankylosing spondylitis, psoriatic arthritis or Reiter's disease);
(iii) a muscular condition such as polymyositis or myasthenia gravis;
(iv) a gastrointestinal tract condition such as inflammatory bowel disease (including Crohn's disease and ulcerative colitis), colitis, gastric ulcer, Coeliac disease, proctitis, pancreatitis, eosinopilic gastro-enteritis, mastocytosis, antiphospholipid syndrome, or a food-related allergy which may have effects remote from the gut (e.g., migraine, rhinitis or eczema);
(v) a respiratory system condition such as chronic obstructive pulmonary disease (COPD), asthma (including eosinophilic, bronchial, allergic, intrinsic, extrinsic or dust asthma, and particularly chronic or inveterate asthma, such as late asthma and airways hyper-responsiveness), bronchitis, rhinitis (including acute rhinitis, allergic rhinitis, atrophic rhinitis, chronic rhinitis, rhinitis caseosa, hypertrophic rhinitis, rhinitis pumlenta, rhinitis sicca, rhinitis medicamentosa, membranous rhinitis, seasonal rhinitis e.g. hay fever, and vasomotor rhinitis), sinusitis, idiopathic pulmonary fibrosis (IPF), sarcoidosis, farmer's lung, silicosis, asbestosis, volcanic ash induced inflammation, adult respiratory distress syndrome, hypersensitivity pneumonitis, or idiopathic interstitial pneumonia; (vi) a vascular condition such as atherosclerosis, Behcet's disease, vasculitides, or Wegener's granulomatosis;
(vii) an autoimmune condition such as systemic lupus erythematosus, Sjögren's syndrome, systemic sclerosis, Hashimoto's thyroiditis, type I diabetes, idiopathic thrombocytopenia purpura, or Graves disease;
(viii) an ocular condition such as uveitis, allergic conjunctivitis, or vernal conjunctivitis;
(ix) a nervous condition such as multiple sclerosis or encephalomyelitis;
(x) an infection or infection-related condition, such as Acquired Immunodeficiency Syndrome (AIDS), acute or chronic bacterial infection, acute or chronic parasitic infection, acute or chronic viral infection, acute or chronic fungal infection, meningitis, hepatitis (A, B or C, or other viral hepatitis), peritonitis, pneumonia, epiglottitis, malaria, dengue hemorrhagic fever, leishmaniasis, streptococcal myositis, Mycobacterium tuberculosis (including Mycobacterium tuberculosis and HIV co-infection), Mycobacterium avium intracellulare, Pneumocystis carinii pneumonia, orchitis/epidydimitis, legionella, Lyme disease, influenza A, Epstein-Barr virus infection, viral encephalitis/aseptic meningitis, or pelvic inflammatory disease;
(xi) a renal condition such as mesangial proliferative glomerulonephritis, nephrotic syndrome, nephritis, glomerular nephritis, obesity related glomerulopathy, acute renal failure, acute kidney injury, uremia, nephritic syndrome, kidney fibrosis including chronic crystal nephropathy, or renal hypertension;
(xii) a lymphatic condition such as Castleman's disease;
(xiii) a condition of, or involving, the immune system, such as hyper IgE syndrome, lepromatous leprosy, familial hemophagocytic lymphohistiocytosis, or graft versus host disease;
(xiv) a hepatic condition such as chronic active hepatitis, non-alcoholic steatohepatitis (NASH), alcohol-induced hepatitis, non-alcoholic fatty liver disease (NAFLD), alcoholic fatty liver disease (AFLD), alcoholic steatohepatitis (ASH), primary biliary cirrhosis, fulminant hepatitis, liver fibrosis, or liver failure;
(xv) a cancer, including those cancers listed above;
(xvi) a burn, wound, trauma, haemorrhage or stroke;
(xvii) radiation exposure;
(xviii) a metabolic disease such as type 2 diabetes (T2D), atherosclerosis, obesity, gout or pseudo-gout; and/or
(xix) pain such as inflammatory hyperalgesia, pelvic pain, allodynia, neuropathic pain, or cancer-induced bone pain.

An embodiment of the present invention is a compound according to formula I as described herein for the treatment or prophylaxis of a disease, disorder or condition selected from:
(i) inflammation;
(ii) an auto-immune disease;
(iii) cancer;
(iv) an infection;
(v) a central nervous system disease;
(vi) a metabolic disease;
(vii) a cardiovascular disease;
(viii) a respiratory disease;
(ix) a liver disease;
(x) a renal disease;
(xi) an ocular disease;
(xii) a skin disease;
(xiii) a lymphatic condition;
(xiv) a psychological disorder;
(xv) graft versus host disease;
(xvi) allodynia;
(xvii) a condition associated with diabetes; and
(xviii) any disease where an individual has been determined to carry a germline or somatic non-silent mutation in NLRP3.

An embodiment of the present invention is the use of a compound according to formula Ib as described herein in the treatment or prophylaxis of a disease, disorder or condition, wherein the disease, disorder or condition is responsive to NLRP3 inhibition.

An embodiment of the present invention is the use of a compound according to formula Ib as described herein in the treatment or prophylaxis of a disease, disorder or condition selected from Alzheimer's disease and Parkinson's disease.

An embodiment of the present invention is the use a compound according to formula Ib as described herein for use in the treatment or prophylaxis of a disease, disorder or condition selected from Asthma or COPD.

An embodiment of the present invention is a compound according to formula Ib as described herein for the treatment or prophylaxis of a disease, disorder or condition selected from Alzheimer's disease and Parkinson's disease.

An embodiment of the present invention is a compound according to formula Ib as described herein for the treatment or prophylaxis of a disease, disorder or condition selected from Asthma or COPD.

An embodiment of the present invention is the use of a compound according to formula Ib as described herein for preparation of a medicament for the treatment or prophylaxis of a disease, disorder or condition selected from Alzheimer's disease and Parkinson's disease.

An embodiment of the present invention is the use of a compound according to formula Ib as described herein for the preparation of a medicament for the treatment or prophylaxis of a disease, disorder or condition selected from Asthma or COPD.

An embodiment of the present invention is a method of treatment or prophylaxis of a disease, disorder or condition selected from Alzheimer's disease and Parkinson's disease, which method comprises administering an effective amount of a compound according to formula Ib as described herein.

An embodiment of the present invention is a method of treatment or prophylaxis of a disease, disorder or condition selected from Asthma or COPD, which method comprises administering an effective amount of a compound according to formula Ib as described herein.

An embodiment of the present invention relates to a method of inhibiting NLRP3, which method comprises administering an effective amount of a compound according to formula Ib as described herein.

Also an embodiment of the present invention are compounds of formula Ib as described herein, when manufactured according to any one of the described processes.

An embodiment of the present invention is a pharmaceutical composition comprising a compound according to formula Ib as described herein and a therapeutically inert carrier.

An embodiment of the present invention is the use of a compound according to formula I as described herein in the treatment or prophylaxis of a disease, disorder or condition, wherein the disease, disorder or condition is responsive to NLRP3 inhibition.

An embodiment of the present invention is the use of a compound according to formula I as described herein in the treatment or prophylaxis of a disease, disorder or condition selected from Alzheimer's disease and Parkinson's disease.

An embodiment of the present invention is the use a compound according to formula I as described herein for use in the treatment or prophylaxis of a disease, disorder or condition selected from Asthma or COPD.

An embodiment of the present invention is a compound according to formula I as described herein for the treatment or prophylaxis of a disease, disorder or condition selected from Alzheimer's disease and Parkinson's disease.

An embodiment of the present invention is a compound according to formula I as described herein for the treatment or prophylaxis of a disease, disorder or condition selected from Asthma or COPD.

An embodiment of the present invention is the use of a compound according to formula I as described herein for preparation of a medicament for the treatment or prophylaxis of a disease, disorder or condition selected from Alzheimer's disease and Parkinson's disease.

An embodiment of the present invention is the use of a compound according to formula I as described herein for the preparation of a medicament for the treatment or prophylaxis of a disease, disorder or condition selected from Asthma or COPD.

An embodiment of the present invention is a method of treatment or prophylaxis of a disease, disorder or condition selected from Alzheimer's disease and Parkinson's disease, which method comprises administering an effective amount of a compound according to formula I as described herein.

An embodiment of the present invention is a method of treatment or prophylaxis of a disease, disorder or condition selected from Asthma or COPD, which method comprises administering an effective amount of a compound according to formula I as described herein.

An embodiment of the present invention relates to a method of inhibiting NLRP3, which method comprises administering an effective amount of a compound according to formula I as described herein.

Also an embodiment of the present invention are compounds of formula I as described herein, when manufactured according to any one of the described processes.

An embodiment of the present invention is a pharmaceutical composition comprising a compound according to formula I as described herein and a therapeutically inert carrier.

### Assay Procedures

### NLRP3 and Pyroptosis

It is well established that the activation of NLRP3 leads to cell pyroptosis and this feature plays an important part in the manifestation of clinical disease (Yan-gang Liu et al., Cell Death & Disease, 2017, 8(2), e2579; Alexander Wree et al., Hepatology, 2014, 59(3), 898-910; Alex Baldwin et al., Journal of Medicinal Chemistry, 2016, 59(5), 1691-1710; Ema Ozaki et al., Journal of Inflammation Research, 2015, 8, 15-27; Zhen Xie & Gang Zhao, Neuroimmunology Neuroinflammation, 2014, 1(2), 60-65; Mattia Cocco et al., Journal of Medicinal Chemistry, 2014, 57(24), 10366-10382; T. Satoh et al., Cell Death & Disease, 2013, 4, e644). Therefore, it is anticipated that inhibitors of NLRP3 will block pyroptosis, as well as the release of pro-inflammatory cytokines (e.g. IL-1β) from the cell.

### THP-1 Cells: Culture and Preparation

THP-1 cells (ATCC #TIB-202) were grown in RPMI containing L-glutamine (Gibco #11835) supplemented with 1 mM sodium pyruvate (Sigma #S8636) and penicillin (100 units/ml)/streptomycin (0.1 mg/ml) (Sigma #P4333) in 10% Fetal Bovine Serum (FBS) (Sigma #F0804). The cells were routinely passaged and grown to confluency (˜10⁶ cells/ml). On the day of the experiment, THP-1 cells were harvested and resuspended into RPMI medium (without FBS). The cells were then counted and viability (>90%) checked by Trypan blue (Sigma #T8154). Appropriate dilutions were made to give a concentration of 625,000 cells/ml. To this diluted cell solution was added LPS (Sigma #L4524) to give a 1 μg/ml Final Assay Concentration (FAC). 40 μl of the final preparation was aliquoted into each well of a 96-well plate. The plate thus prepared was used for compound screening.

### THP-1 Cells Pyroptosis Assay

The following method step-by-step assay was followed for compound screening.
1. Seed THP-1 cells (25,000 cells/well) containing 1.0 μg/ml LPS in 40 μl of RPMI medium (without FBS) in 96-well, black walled, clear bottom cell culture plates coated with poly-D-lysine (VWR #734-0317)
2. Add 5 μl compound (8 points half-log dilution, with 10 μM top dose) or vehicle (DMSO 0.1% FAC) to the appropriate wells
3. Incubate for 3 hours at 37° C., 5% CO₂
4. Add 5 μl nigericin (Sigma #N7143) (FAC 5 μM) to all wells
5. Incubate for 1 hr at 37° C., 5% CO₂
6. At the end of the incubation period, spin plates at 300×g for 3 mins and remove supernatant
7. Then add 50 μl of resazurin (Sigma #R7017) (FAC 100 μM resazurin in RPMI medium without FBS) and incubate plates for a further 1-2 hours at 37° C. and 5% CO₂
8. Plates were read in an Envision reader at Ex 560 nm and Em 590 nm
9. IC₅₀ data is fitted to a non-linear regression equation (log inhibitor vs response-variable slope 4-parameters)

The results of the pyroptosis assay are summarised in Table 1 below as THP IC₅₀.

### Human Whole Blood IL-1β Release Assay

For systemic delivery, the ability to inhibit NLRP3 when the compounds are present within the bloodstream is of great importance. For this reason, the NLRP3 inhibitory activity of a number of compounds in human whole blood was investigated in accordance with the following protocol.

Human whole blood in Li-heparin tubes was obtained from healthy donors from a volunteer donor panel.
1. Plate out 80 μl of whole blood containing 1 μg/ml of LPS in 96-well, clear bottom cell culture plate (Corning #3585)
2. Add 10 μl compound (8 points half-log dilution with 10 μM top dose) or vehicle (DMSO 0.10% FAC) to the appropriate wells
3. Incubate for 3 hours at 37° C., 5% CO₂
4. Add 10 μl nigericin (Sigma #N7143) (10 μM FAC) to all wells
5. Incubate for 1 hr at 37° C., 5% CO₂
6. At the end of the incubation period, spin plates at 300×g for 5 mins to pellet cells and remove 20 μl of supernatant and add to 96-well v-bottom plates for IL-1β analysis (note: these plates containing the supernatants can be stored at −80° C. to be analysed at a later date)
7. IL-1β was measured according to the manufacturer protocol (Perkin Elmer-AlphaLisa IL-1 Kit AL220F-5000)
8. IC₅₀ data is fitted to a non-linear regression equation (log inhibitor vs response-variable slope 4-parameters)

The results of the human whole blood assay are summarised in Table 1 below as HWB IC₅₀.

hERG Screening Assay

In the drug development process of small molecules, one of the most frequent adverse side effects, leading to the failure of drugs, is the cardiac arrhythmias. Such failure is often related to the capacity of the drug to inhibit the human ether-à-go-go-related gene (hERG) cardiac potassium channel. Having no or low inhibition of the hERG cardiac potassium channel is therefore considered as beneficial.

### Cells

The CHO crelox hERG cell line (ATCC reference Nr. PTA-6812, female Chinese hamster cells) was generated and validated at Roche. Ready-to-use frozen instant CHO-hERG cells were cryopreserved at Evotec (Germany) and used directly in the experiments.

### Experimental Solutions

The extracellular solution contains (in mM): NaCl 150; KCl 4; CaCl₂ 1; MgCl₂ 1; HEPES 10; pH 7.2-7.4 with NaOH, osmolarity 290-330 mOsm. The internal solution contains (in mM): KCl, 10; KF, 100; NaCl, 10; HEPES, 10; EGTA, 20; pH=7.0-7.4 with KOH, osmolarity 260-300 mOsm.

### Electrophysiology

The effects of a compound on hERG K+-currents parameters will be evaluated at 2 concentrations in at least 4 cells.

The hERG test is performed using automated patch clamp system SynchroPatch® 384 (Nanion Technologies GmbH, Germany). K+ currents are measured with the patch-voltage-clamp technique in the whole-cell configuration at 35-37° C.

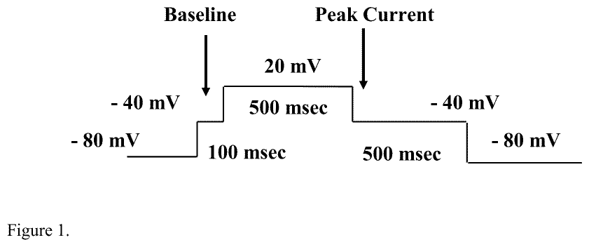

Cells were held at a resting voltage of −80 mV and they were stimulated by a voltage pattern shown in FIG. 1 (pulse pattern used to elicit outward K⁺ current at 35-37° C.) to activate hERG channels and conduct outward IKhERG current, at a stimulation frequency of 0.1 Hz (6 bpm)

### Data Analysis

The amplitudes of IKhERG were recorded in each concentration of drug and they were compared to the vehicle control values (taken as 100%) to define fractional blocks. The concentration-response data were fitted with the following relationship:

| | $I ( C ) = \frac{100}{1 + {( C / IC 50 )}^{h}}$ |
|---|---|
| where | C is the concentration, |
| | IC₅₀ is the concentration producing 50% block |
| | h is the Hill coefficient. |

Concentration-response curves were fitted by non-linear regression analysis using EworkBook suite (ID Business Solutions Ltd, UK). Data fit was done with the 4 Parameter Logistic Model (fit=(A+(B/(1+((x/C)\hat{ }D)))), where A=0 and B=100).

### Transcellular P-gp Assay:

The general assay uses transfected LLC-PKT cells (porcine kidney epithelial cells) over-expressing human or mouse P-gp, cultured on 96 well semi-permeable filter membrane plates, where they form a polarized monolayer with tight junctions, and act as a barrier between the apical and basolateral compartment.

P-gp is expressed in the apical-facing membrane of the monolayer.

The tightness of the cell monolayer and functional activity of P-gp are confirmed by addition of a cell-impermeable marker, Lucifer yellow, and a reference P-gp substrate, edoxaban, respectively.

### PAMPA:

PAMPA (Parallel Artificial Membrane Permeability Assay) is a first line permeability screen for drug candidates. The PAMPA assay mimics the transcellular absorption conditions using an artificial phospholipid membrane. This assay determines a permeability value that can be used for compound optimization and ranking purposes as well as input parameters for in silico models to predict intestinal absorption.

The donor concentration is measured at t-start (reference) and compared with the donor and acceptor concentration after a certain time (t-end) to calculate the extent of passage of the compound through the membrane.

### Microsomal Stability:

Incubations of test compounds at 1 μM in microsomes (0.5 mg/mL) plus cofactor NADPH are performed in 96 well plates at 37° C. on a TECAN (Tecan Group Ltd, Switzerland) automated liquid handling system. After a 10 minutes pre-incubation step of the test compound with the microsomes, the enzymatic reaction is started by the addition of cofactors. At 1, 3, 6, 9, 15, 25, 35 and 45 minutes, aliquots of the incubations are removed and quenched with 1:3 (v/v) acetonitrile containing internal standard. Samples are then cooled and centrifuged before analysis of the supernatant by LC-MS/MS 2.

### Metabolic Stability in Hepatocytes:

### Assay Descriptions:

Biological materials. Cryopreserved hepatocytes [mouse, rat, rabbit, monkey and human (male and female; mixed)] are obtained. Viability of hepatocytes after reconstitution is at least 80% throughout the study. Ready-to-use rat/human HepatoPac® cultures [long-term hepatocyte co-cultures; pooled (n=5 for male and n=5 for female for human)] with stromal mouse fibroblasts (negative control; pooled) with the plates for incubations, application medium and maintenance medium are acquired.

Metabolism by suspended hepatocytes. Primary pooled cryopreserved hepatocytes are reconstituted in pre-warmed William's E media containing 10% FCS, 0.05 mg/mL streptomycin and 50 U/mL penicillin and 0.4 mM L-glutamine; and 0.01 mg/mL gentamicin, 0.048 mg/mL hydrocortisone and 0.004 mg/mL insulin, to a final suspension density of 1×106 cells/mL. The incubation was performed fully automatically with Liquid Handling System (Tecan) equipped with a CO2 incubator with an orbital shaker. After the addition of a test compound at e.g. 1 μM to the wells (1×105 cells/well), the 96-well hepatocyte suspension culture plates are incubated in a 5% CO2 at 37° C. Samples are quenched by addition of acetonitrile (including an internal standard) to the incubation well at the designated time points up to 2 h.

Metabolism by HepatoPac®. Incubations for a test article (at e.g. 1 μM, 0.1% v/v DMSO) as conducted in suspension assays are performed in 96-well plates containing either a co-culture of adherent hepatocytes with mouse fibroblast control cells or control cells alone (5% CO2 atmosphere and 37° C.). The incubation media in human HepatoPac® is identical with that in suspended hepatocytes. At defined time points (2, 18, 26, 48, 72 and 96 h), whole wells are quenched with ice-cold acetonitrile containing an internal standard.

Samples are then centrifuged appropriately and the supernatant analyzed by LC-MS/MS. The incubation is conducted in n=1 or 2.

TABLE 1
NLRP3 inhibitory activity

| | THP-1 pyroptosis | Human whole blood IB-1β |
|---|---|---|
| | Assay | Assay IC50 |
| Example No. | IC₅₀ (nM) | (nM) |
| 1 | 1.6 | 0.088 |
| 2 | 3.5 | 0.349 |
| 3 | 57.2 | 0.091 |
| 4 | 52.4 | 0.328 |
| 5-TFA | 12.4 | 0.028 |
| 5-HCl | 11.6 | |
| 6 | 3.5 | 0.028 |
| 7 | 9.1 | 0.592 |
| 9 | 2.7 | 0.217 |
| 10 | 2.3 | 0.024 |
| 11 | 76.0 | |
| 12 | 2.2 | 0.014 |
| 13 | 18.0 | 2.6 |
| 14 | 7.8 | 0.111 |
| 15 | 8.6 | 0.232 |
| 16 | 361.5 | |
| 17 | 3.6 | 0.015 |
| 18 | 2.2 | 0.033 |
| 19 | 46.6 | 0.30 |

The invention will now be illustrated by the following examples which have no limiting character.

In case the preparative examples are obtained as a mixture of enantiomers or diastereoisomers, the pure enantiomers or diastereomers can be obtained by methods described herein or by methods known to those skilled in the art, such as e.g. chiral chromatography or crystallization.

### Experimental Methods

### Abbreviations

| | |
|---|---|
| ACN | acetonitrile |
| CH₂Cl₂ | dichloromethame |
| DIPEA | diisopropylethylamine |
| DMF | N,N-dimethylformamide |
| DMSO | Dimethyl sulfoxide |
| EtOAc | Ethyl acetate |
| h, hrs | hour, hours |
| HPLC | High-performance liquid chromatography |
| min(s) | Minute(s) |
| MSD | mass selective detector |
| NMR | Nuclear magnetic resonance spectroscopy |
| NMP | N-methyl-2-pyrrolidinone |
| prep | preparative |
| PE | Petrol ether |
| RP | Reverse phase |
| TFA | Trifluoroacetic acid |
| TLC | Thin-layer chromatography |

### Analytical Methods

NMR spectra were run on Bruker 400 MHz spectrometers using ICON-NMR, under TopSpin program control. Spectra were measured at 298 K, unless indicated otherwise, and were referenced relative to the solvent resonance.

### LC-MS Methods

Using SHIMADZU LCMS-2020, Agilent 1200 LC/G1956A MSD and Agilent 1200\G6110A, Agilent 1200 LC & Agilent 6110 MSD. Mobile Phase: A: 0.025% NH₃·H₂O in water (v/v); B: acetonitrile. Column: Kinetex EVO C₁₈ 2.1×30 mm, 5 μm.

### Systems

### Waters Acquity UPLC

Binary Pump
Autosampler Waters 2777C (alias CTC Pal HT)
Column Managers (4 Columns)
Photodiode Array Detector (PDA)
Single Quadrupole Mass Spectrometer (SQD1 respectively SQD2)

### Eluents

Channel A: Water 0.10% Formic Acid
Channel B: Acetonitrile 0.07% Formic Acid

### Built-In Columns (@ 50° C.):

Column 1: Agilent Zorbax Eclipse Plus C18, Rapid Resolution HT, 2.1×30 mm, 1.8 μm, Part. no. 959731-902
Column 2: (only for MS1+5+7): Waters Acquity UPLC BEH C18, 2.1×50 mm, 1.7 μm, Part. no. 186002350
Column 3: none
Column 4: none (Flow injection)

### Methods

Fast_Gradient (2 min. Column 1, Mass range m/z 150-900)

| Time [min] | Flow Rate [ml/min] | % A | % B |
|---|---|---|---|
| Initial | 0.8 | 97 | 3 |
| 0.2 | 1.0 | 97 | 3 |
| 1.7 | 1.0 | 3 | 97 |
| 2.0 | 1.0 | 3 | 97 |
| 2.1 | 1.0 | 97 | 3 |

### EXAMPLES

All examples and intermediates were prepared under nitrogen atmosphere if not specified otherwise.

### Examples 1 and 2

### (rac)-2-[7-[(1-Ethyl-3-piperidyl)amino]thieno[2,3-d]pyridazin-4-yl]-5(trifluoromethyl)phenol; formic acid

### and

### (rac)-2-[4-[(1-Ethyl-3-piperidyl)amino]thieno[2,3-d]pyridazin-7-yl]-5 (trifluoromethyl)phenol; formic acid

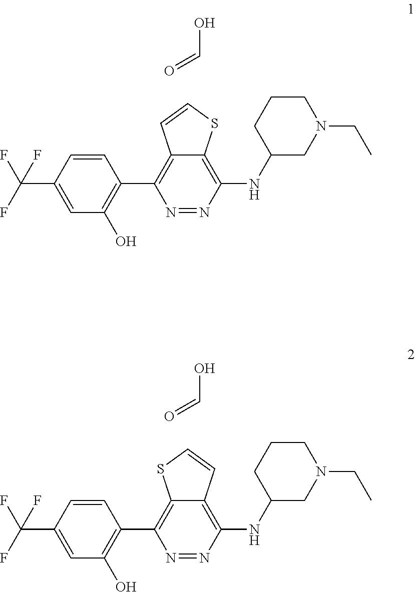

### Step A: (rac)-7-Chloro-N-(1-ethyl-3-piperidyl)thieno[2,3-d]pyridazin-4-amine and (rac)-4-Chloro-N-(1-ethyl-3-piperidyl)thieno[2,3-d]pyridazin-7-amine

To a mixture of 4,7-dichlorothieno[2,3-d]pyridazine (CAS #699-89-8, 50 mg, 0.244 mmol, 1.0 eq) and (rac)-1-ethylpiperidin-3-amine (CAS #6789-94-2, 41.8 μL, 0.293 mmol, 1.2 eq) in DMSO (0.2 mL) was added DIPEA (128 μL, 0.731 mmol, 3.0 eq). The reaction mixture was heated to 120° C. for 16 hrs. The yellow reaction mixture was extracted with ethyl acetate and water. The organic layer was washed with brine. The aqueous layers were back extracted twice with ethyl acetate. The combined organic layers were dried over sodium sulfate, filtered and concentrated in vacuo. The crude product (brown oil, 110.9 mg) was combined with another crude mixture from a different experiment done on the same scale in a different solvent (50 mg in NMP) giving a new crude residue (orange oil, 169 mg) which was adsorbed on ISOLUTE HM-N and purified by flash chromatography (silica gel, 4 g, CH₂Cl₂ with MeOH gradient) to afford the title compounds (82.5 mg) as a dark yellow solid containing a mixture of regioisomers. LCMS: m/z 297.1 [M+H]⁺, ESI pos.

### Step B: (rac)-2-[7-[(1-Ethyl-3-piperidyl)amino]thieno[2,3-d]pyridazin-4-yl]-5-(trifluoromethyl)phenol; formic acid and (rac)-2-[4-[(1-ethyl-3-piperidyl)amino]thieno[2,3-d]pyridazin-7-yl]-5-(trifluoromethyl)phenol; formic acid

A mixture of regioisomers of aforementioned (rac)-7-chloro-N-(1-ethyl-3-piperidyl)thieno[2,3-d]pyridazin-4-amine and (rac)-4-chloro-N-(1-ethyl-3-piperidyl)thieno[2,3-d]pyridazin-7-amine (82.5 mg, 0.278 mmol, 1.0 eq), [2-hydroxy-4-(trifluoromethyl)phenyl]boronic acid (CAS #1072951-50-8, 97.0 mg, 0.471 mmol, 1.7 eq), potassium carbonate (183.6 mg, 1.33 mmol, 4.8 eq) and 1,1′-bis(diphenylphosphino)ferrocene-palladium(i) dichloride dichloromethane complex (26.2 mg, 0.032 mmol, 0.116 eq) in 1,4-dioxane (1.5 mL) and water (0.7 mL) was flushed with argon and stirred at 90° C. for 2 hrs. The reaction mixture was cooled to room temperature; MeOH was added (2 mL) and concentrated in vacuo. The crude product was purified by RP prep-HPLC (column: YMC-Triart Cis, 12 nm, 5 μm, 100×30 mm; condition: ACN/water+0.1% HCOOH, 11 mins run time, gradient 10-98-100 ACN in water) to afford the title compound 1 (14.8 mg, 11% yield) as a yellow solid and its regioisomer 2 (29.5 mg, 23% yield) both as format salt in a molar ratio respectively (in molar following to NMR product:formate) 53:47 and 52:48. LCMS: m/z 423.1 [M+H]⁺, ESI pos.

### Examples 3 and 4

### (rac)-N-(1-ethyl-3-piperidyl)-4-[4-(trifluoromethyl)phenyl]thieno[2,3-d]pyridazin-7-amine

### and

### (rac)-N-(1-ethyl-3-piperidyl)-7-[4-(trifluoromethyl)phenyl]thieno[2,3-d]pyridazin-4-amine

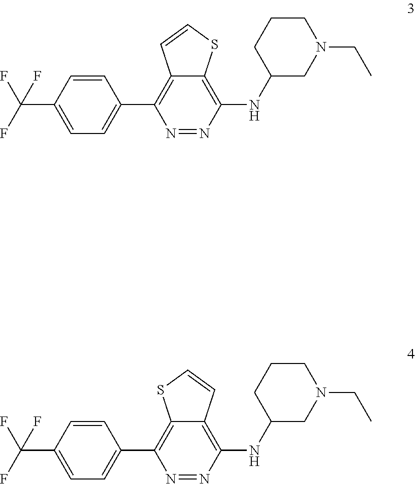 [SMILES: CCN1CCCC(Nc2nnc(-c3ccc(C(F)(F)F)cc3)c3ccsc23)C1 ; CCN1CCCC(Nc2nnc(-c3ccc(C(F)(F)F)cc3)c3sccc23)C1]

Similarly to previous example 1 and 2, step B, a mixture of regioisomers of aforementioned (rac)-7-chloro-N-(1-ethyl-3-piperidyl)thieno[2,3-d]pyridazin-4-amine and (rac)-4-chloro-N-(1-ethyl-3-piperidyl)thieno[2,3-d]pyridazin-7-amine (80 mg, 0.270 mmol, 1.0 eq), [4-(trifluoromethyl)phenyl]boronic acid (CAS #128796-39-4, 86.8 mg, 0.457 mmol, 1.7 eq), potassium carbonate (178 mg, 1.29 mmol, 4.8 eq) and 1,1′-bis(diphenylphosphino)ferrocene-palladium(II) dichloride dichloromethane complex (25.4 mg, 0.031 mmol, 0.116 eq) in 1,4-dioxane (1.6 mL) and water (0.8 mL) was flushed with argon and stirred at 90° C. for 2 hrs. The reaction mixture was cooled to room temperature and then was extracted with ethyl acetate and water. The organic layer was washed with brine. The aqueous layers were back extracted twice with ethyl acetate. The combined organic layers were dried over sodium sulfate, filtered and concentrated in vacuo. The crude product was purified by RP prep-HPLC (column: YMC-Triart Cis, 12 nm, 5 μm, 100×30 mm; condition: ACN/water+0.1% triethylamine,) to afford the title compound 3 (28.1 mg, 24% yield) as a yellow solid and its regioisomere 4 (47.9 mg, 42% yield). LCMS: m/z 407.2 [M+H]⁺, ESI pos.

### Example 5-HCl

### 2-[4-[[(3R)-1-Ethyl-3-piperidyl]amino]-1-methyl-pyrrolo[2,3-d]pyridazin-7-yl]-5-(trifluoromethyl)phenol; hydrochloride

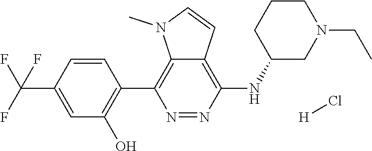 [SMILES: CCN1CCC[C@@H](Nc2nnc(-c3ccc(C(F)(F)F)cc3O)c3c2ccn3C)C1 ; Cl]

### Step A: methyl 1-(2-trimethylsilylethoxymethyl)pyrrole-3-carboxylate

To a mixture of methyl 1H-pyrrole-3-carboxylate (CAS #2703-17-5, 10.0 g, 79.9 mmol, 1.0 eq) in DMF (100 mL) was added NaH (3.52 g, 87.9 mmol, 1.1 eq, 60% purity) under N₂ at 0° C. The mixture was stirred for 15 mins, then 2-(trimethylsilyl)ethoxymethyl chloride (18.4 mL, 104 mmol, 1.3 eq) was added and stirred for 45 mins at 25° C. TLC (PE/EtOAc=2:1) showed the reaction was completed and one new spot was detected. The mixture was poured into water (20 mL) and extracted with ethyl acetate (100 mL*3). The combined organic layers were washed with brine (20 mL), dried over anhydrous sodium sulfate, filtered, and the filtrate was concentrated under reduced pressure. The residue was purified by column silica gel using PE/EtOAc=2/1 to afford the title compound (20 g, 98% yield) as colorless oil. LCMS: m/z 255.9 [M+H]⁺, ESI.

### Step B: Methyl 2-[hydroxy-[2-methoxy-4-(trifluoromethyl)phenyl]methyl]-1-(2-trimethylsilylethoxymethyl)pyrrole-3-carboxylate

A mixture of aforementioned methyl 1-(2-trimethylsilylethoxymethyl)pyrrole-3-carboxylate (5.0 g, 19.6 mmol, 1.0 eq) in diisopropyl ether (100 mL) was added Lithium diisopropylamide (24.5 mL, 48.9 mmol, 2.5 eq) dropwise under N₂ at −60° C. and stirred for 10 mins. Then the 2-methoxy-4-(trifluoromethyl)benzaldehyde (4.40 g, 21.5 mmol, 1.1 eq) was added and the mixture was stirred for 50 mins at −60° C. After reaction completion (TLC:PE/EtOAc=10:1), the mixture was quenched with sat. NH₄Cl (10 mL). The aqueous layer and extracted with EtOAc (100 mL*2). The combined organic layers were washed with water (50 mL) and dried over anhydrous sodium sulfate, filtrated and concentrated under reduced pressure. The crude product was purified by column chromatography on silica gel eluted with PE/EtOAc=10:1 to afford the title compound (4.50 g, 50% yield) as colorless oil. ¹H NMR (400 MHz, METHANOL-d₄) 6=7.49 (d, 1H), 7.25 (d, 1H), 7.20 (d, 1H), 6.79 (d, 1H), 6.73 (s, 1H), 6.57 (d, 1H), 5.31-5.21 (m, 2H), 3.83, 3.81 (2 s, 3H each), 3.26-3.10, 0.64-0.55, 0.50-0.41 (3 m, 2H, 1H, 1H), −0.10 (s, 9H).

### Step C: Methyl 2-[2-methoxy-4-(trifluoromethyl)benzoyl]-1-(2-trimethylsilylethoxymethyl)pyrrole-3-carboxylate

A mixture of aforementioned methyl 2-[hydroxy-[2-methoxy-4-(trifluoromethyl)phenyl]methyl]-1-(2-trimethylsilylethoxymethyl)pyrrole-3-carboxylate (4.50 g, 9.79 mmol, 1.0 eq) and 2,2-dimethoxypropane (6.23 g, 14.7 mmol, 1.5 eq) in CH₂Cl₂ (50 mL) was stirred for 1 h at 25° C. Upon reaction completion (TLC:PE/EtOAc=10:1), the mixture was quenched with sat. Na₂SO₃ (50 mL) and the aqueous layer was extracted with CH₂Cl₂ (100 mL*2). The combined organic layers were washed with water (50 mL) and dried over anhydrous sodium sulfate, filtrated and the filtrate was concentrated under reduced pressure. The crude product was purified by column chromatography on silica gel eluted with PE/EtOAc=10:1 to afford the title compound (2.0 g, 44% yield) as colorless oil. LCMS: m/z 458.0 [M+H]⁺, ESI.

### Step D: 7-[2-Methoxy-4-(trifluoromethyl)phenyl]-1-(2-trimethylsilylethoxymethyl)-5H-pyrrolo[2,3-d]pyridazin-4-one

A mixture of aforementioned methyl 2-[2-methoxy-4-(trifluoromethyl)benzoyl]-1-(2-trimethylsilylethoxymethyl)pyrrole-3-carboxylate (1.50 g, 3.28 mmol, 1.0 eq) and hydrazine hydrate (820 mg, 16.4 mmol, 5.0 eq) in ethanol (10 mL) was stirred for 16 hrs at 70° C. After reaction completion, the mixture was poured into water (20 mL) and extracted with EtOAc (100 mL*3). The combined organic layers were washed with brine (20 mL), dried over anhydrous sodium sulfate, filtered, and concentrated under reduced pressure. The residue was purified by column silica gel using PE/EtOAc=2/1 to afford the title compound (1.0 g, 68% yield) as a white solid. LCMS: m/z 440.4 [M+H]⁺, ESI.

### Step E: 4-Chloro-7-[2-methoxy-4-(trifluoromethyl)phenyl]-1H-pyrrolo[2,3-d]pyridazine

A mixture of aforementioned 7-[2-methoxy-4-(trifluoromethyl)phenyl]-1-(2-trimethylsilylethoxymethyl)-5H-pyrrolo[2,3-d]pyridazin-4-one (1.0 g, 2.28 mmol, 1.0 eq) and POCl₃ (3.49 g, 22.8 mmol, 10 eq) in toluene (10 mL) was stirred for 2 hrs at 110° C. After reaction completion, the mixture was concentrated under reduced pressure. The crude product was purified by reversed-phase flash (CombiFlash 0.1% TFA aqueous-MeCN condition) and followed by lyophilization to afford the title compound (340 mg, 46% yield) as a white solid. LCMS: m/z 327.9 [M+H]⁺, ESI.

### Step F: 4-Chloro-7-[2-methoxy-4-(trifluoromethyl)phenyl]-1-methyl-pyrrolo[2,3-d]pyridazine

To a mixture of aforementioned 4-chloro-7-[2-methoxy-4-(trifluoromethyl)phenyl]-1H-pyrrolo[2,3-d]pyridazine (340 mg, 1.04 mmol, 1.0 eq) in DMF (4 mL) was added NaH (62.3 mg, 1.56 mmol, 1.5 eq, 60% purity) at 0° C. under N₂ and stirred for 5 mins. Then methyl iodide (221 mg, 1.56 mmol, 1.5 eq) was added to the mixture and stirred for 55 mins at 25° C. Upon reaction completion, the mixture was poured into sat. NH₄Cl solution (20 mL) and extracted with ethyl acetate (100 mL*3). The combined organic layers were washed with brine (20 mL), dried over anhydrous sodium sulfate, filtered, and the filtrate was concentrated under reduced pressure. The residue was purified by column silica gel using PE/EtOAc=4/1 to afford the title compound (260 mg, 67% yield) as a white solid. LCMS: m/z 341.8 [M+H]⁺, ESI.

### Step G: N-[(3R)-1-Ethyl-3-piperidyl]-7-[2-methoxy-4-(trifluoromethyl)phenyl]-1-methyl-pyrrolo[2,3-d]pyridazin-4-amine

To a solution of aforementioned 4-chloro-7-[2-methoxy-4-(trifluoromethyl)phenyl]-1-methyl-pyrrolo [2,3-d]pyridazine (100 mg, 0.29 mmol, 1.0 eq) in 1,4-dioxane (4 mL) was added BinapPdG₃ (20.0 mg, 0.060 mmol, 0.20 eq), cesium carbonate (190.7 mg, 0.590 mmol, 2.0 eq) and 4-chloro-7-[2-methoxy-4-(trifluoromethyl)phenyl]-1-methyl-pyrrolo[2,3-d]pyridazine (100 mg, 0.29 mmol, 1.0 eq) under N₂. The mixture was stirred at 100° C. for 4 hrs. After reaction completion, the reaction mixture was quenched with water (2 mL) and concentrated under reduce pressure. The residue was purified by reversed phase flash (CombiFlash 0.1% TFA aqueous-ACN condition) and following by lyophilization to afford the title compound (100 mg, 79% yield) as a light yellow solid. LCMS: m/z 434.2 [M+H]⁺, ESI.

### Step H: 2-[4-[[(3R)-1-Ethyl-3-piperidyl]amino]-1-methyl-pyrrolo[2,3-d]pyridazin-7-yl]-5-(trifluoromethyl)phenol; hydrochloride

A mixture of aforementioned N-[(3R)-1-ethyl-3-piperidyl]-7-[2-methoxy-4-(trifluoromethyl)phenyl]-1-methyl-pyrrolo[2,3-d]pyridazin-4-amine (130 mg, 0.30 mmol, 1.0 eq) and BBr₃ (1.13 g, 4.50 mmol, 15 eq) in CH₂Cl₂ (2 mL) was stirred at −60° C. for 10 mins, then stirred at 25° C. for 50 mins. After reaction completion, the pH was adjust to pH˜7 by adding ammoniac, then the mixture was filtered and the filtrate was concentrated under reduce pressure. The crude product was purified by reversed-phase flash (CombiFlash 0.1% HCl aqueous-ACN) and following by lyophilization to afford the title compound (92.7 mg, 66% yield) as a light yellow solid. LCMS: m/z 420.0 [M+H]⁺, ESI.

Similarly, to above, example 5 was also prepared as a TFA salt:

### Example 5-TFA

### 2-[4-[[(3R)-1-ethyl-3-piperidyl]amino]-1-methyl-pyrrolo[2,3-d]pyridazin-7-yl]-5-(trifluoromethyl)phenol; 2,2,2-trifluoroacetic acid

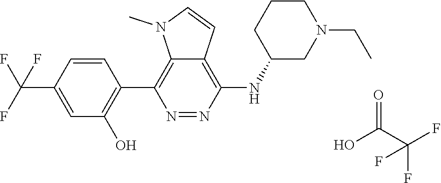

A mixture of aforementioned N-[(3R)-1-ethyl-3-piperidyl]-7-[2-methoxy-4-(trifluoromethyl)phenyl]-1-methyl-pyrrolo[2,3-d]pyridazin-4-amine (30.0 mg, 0.070 mmol, 1 eq) and BBr₃ (173 mg, 0.69 mmol, 10 eq) in CH₂Cl₂ (1 mL) was stirred at −60° C. for 10 mins, then stirred at 25° C. for 50 mins. After reaction completion, the mixture was adjust pH˜7 by addition of ammoniac, then filtered and the filtrate was concentrated under reduce pressure. The crude product was purified by RP flash (CombiFlash 0.1% TFA aqueous-ACN) and following by lyophilization to afford the title compound (15.0 mg, 37% yield) as a light yellow solid. LCMS: m/z 420.1 [M+H]⁺, ESI.

### Example 6

### 2-[4-[[(3R)-1-Ethyl-3-piperidyl]amino]-1-methyl-pyrazolo[3,4-d]pyridazin-7-yl]-5-(trifluoromethyl)phenol; 2,2,2-trifluoroacetic acid

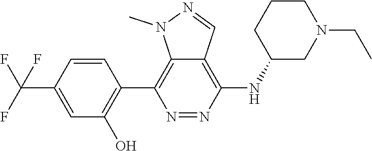 [SMILES: CCN1CCC[C@@H](Nc2nnc(-c3ccc(C(F)(F)F)cc3O)c3c2cnn3C)C1]

### Step A: Diethyl 2-methylpyrazole-3,4-dicarboxylate

To a solution of diethyl 1H-pyrazole-4,5-dicarboxylate (4.00 g, 18.9 mmol, 1.0 eq) in ACN (60 mL) was added potassium carbonate (5.21 g, 37.7 mmol, 2.0 eq). The mixture was stirred at 20° C. for 0.5 hrs, and then iodo methane (4.01 g, 28.3 mmol, 1.5 eq) was added to the mixture. The mixture was stirred at 20° C. for 12 hrs. Upon reaction completion (TLC:PE:EtOAc=3:1), the mixture was quenched with H₂O (100 mL) and extracted with EtOAc (100 mL*3). The organic phase was washed with brine (100 mL*2), dried over anhydrous Na₂SO₄, filtered and the filtrate was concentrated under reduce pressure. The residue was purified by column chromatography (SiO₂, petroleum ether: ethyl acetate, 5:1 to 2:1) to afford the title compound (1.35 g, 32% yield) as a colorless oil. ¹H NMR (400 MHz, DMSO-d₆) δ=7.88 (s, 1H), 4.37, 4.21 (q, 2H each), 3.94 (s, 3H), 1.34-1.21 (m, 6H).

### Step B: 1-Methylpyrazolo[3,4-d]pyridazine-4,7-diol

To a solution of diethyl 2-methylpyrazole-3,4-dicarboxylate (1.1 g, 4.86 mmol, 1.0 eq) in methanol (20 mL) was added hydrazine monohydrate (0.71 mL, 14.6 mmol, 3.0 eq). The mixture was stirred at 20° C. for 24 hrs. The reaction mixture was concentrated under reduce pressure. The residue was purified by RP flash (CombiFlash (0.1% NH₃·H₂O aqueous-ACN condition) and following by lyophilization to afford the title compound (100 mg, 12% yield) as a white solid. LCMS: m/z 167.1 [M+H]⁺, ESI pos.

### Step C: 4,7-Dichloro-1-methyl-pyrazolo[3,4-d]pyridazine

A mixture of aforementioned 1-methylpyrazolo[3,4-d]pyridazine-4,7-diol (200 mg, 1.20 mmol, 1.0 eq) in POCl₃ (2.0 mL) was stirred at 60° C. for 12 hrs. Upon reaction completion, the mixture was concentrated under reduce pressure to afford the title compound (220 mg, 90% yield) as a yellow solid. LCMS: m/z 202.8 [M+H]⁺, ESI pos.

### Step D: 7-Chloro-N-[(3R)-1-ethyl-3-piperidyl]-1-methyl-pyrazolo[3,4-d]pyridazin-4-amine

To a solution of aforementioned 4,7-dichloro-1-methyl-pyrazolo[3,4-d]pyridazine (200 mg, 0.99 mmol, 1.0 eq) and (3R)-1-ethylpiperidin-3-amine (CAS #1020396-26-2, 152 mg, 1.18 mmol, 1.20 eq) in NMP (1 mL) was added potassium carbonate (272 mg, 1.97 mmol, 2.0 eq). The mixture was heated to 85° C. and stirred for 16 hrs under N2. Upon reaction completion, the reaction mixture was cooled to 20° C. and purified by reversed phase flash (CombiFlash 0.1% TFA aqueous-ACN condition) and following by lyophilization to afford the title compound (70 mg, 24% yield) as a yellow solid. LCMS: m/z 295.0 [M+H]⁺, ESI pos.

### Step E: 2-[4-[[(3R)-1-ethyl-3-piperidyl]amino]-1-methyl-pyrazolo[3,4-d]pyridazin-7-yl]-5-(trifluoromethyl)phenol; 2,2,2-trifluoroacetic acid

To a solution of aforementioned 7-chloro-N-[(3R)-1-ethyl-3-piperidyl]-1-methyl-pyrazolo[3,4-d]pyridazin-4-amine (60.0 mg, 0.20 mmol, 1.0 eq) and [2-hydroxy-4-(trifluoromethyl)phenyl]boronic acid (CAS #1072951-50-8, 83.8 mg, 0.410 mmol, 2.0 eq) in 1,4-dioxane (2 mL) and water (0.400 mL) was added CsF (92.8 mg, 0.610 mmol, 3.0 eq) and Pd(dppf)Cl₂ (29.8 mg, 0.040 mmol, 0.20 eq). The above reaction mixture was stirred at 130° C. for 1.5 hrs under microwave. Upon reaction completion, the mixture was concentrated under reduce pressure and purified by reversed phase flash (CombiFlash 0.1% TFA aqueous-ACN condition) and followed by lyophilization to give the crude product. The crude was purified by prep-HPLC (method column 3_Phenomenex Luna C₁₈ 75 mm*30 mm*3 μm; condition water (TFA)-ACN; begin B: 10; End B: 40; gradient time(min): 7; 100% B Hold Time(min): 2; flow rate (mL/min): 25) and followed by lyophilization to afford the title compound (34.4 mg, 30% yield) as a white solid. LCMS: m/z 421.1 [M+H]⁺, ESI pos.

### Purification Method

Automated reversed phase column chromatography was carried out using a Gilson GX-281 system driven by a Gilson-322 pump module, Gilson-156 UV photometer detection unit and Gilson-281 fraction collector.
Phenomenex Luna C₁₈: 75 mm*30 mm*3 μm
pH (water(10 mM TFA)-ACN): 5-6
Average particle size: 3 μm

The column was conditioned before use with 100% ACN (2 min) then brought to 1% ACN (in 0.8 min). Flow rate=25 mL/min.

### Separation Runs:

| | A: water(10 mM | |
|---|---|---|
| Time (min) | TFA | B: MeCN |
| 0 | 90% | 10% |
| 7 | 60% | 40% |
| 7.1 | 0% | 100% |
| 9.1 | 0% | 100% |
| 9.2 | 95% | 5% |
| 9.7 | 95% | 5% |

Detection wavelength: 220 and 254 nm.

Before each new run, the cartridge was cleaned using the conditioning method.

### Example 7

### 2-[4-[[(3R)-1-ethyl-3-piperidyl]amino]furo[2,3-d]pyridazin-7-yl]-5-(trifluoromethyl)phenol

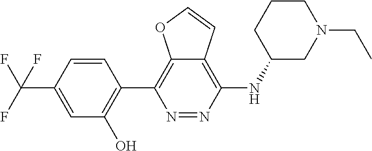 [SMILES: CCN1CCC[C@@H](Nc2nnc(-c3ccc(C(F)(F)F)cc3O)c3occc23)C1]

### Step A: Furo[2,3-d]pyridazine-4,7-diol

To a mixture of dimethyl furan-2,3-dicarboxylate (4.50 g, 24.4 mmol, 1.0 eq) in ethanol (45 mL) was added hydrazine hydrate (1.22 g, 244 mmol, 10 eq). The mixture was stirred at 70° C. for 2 hrs. Then the mixture was filtrated and the filter cake was added to a solution of HCl (3.05 mL, 36.7 mmol, 1.5 eq) in water (30 mL). Upon reaction completion (TLC (PE:EtOAc=1:1)). The mixture was filtrated and a white filter cake was obtained. The filter cake was trituration with water to afford the title compound (2.70 g, 73% yield) as a white solid. ¹H NMR (400 MHz, DMSO-d₆) δ=11.77 (br. s, 2H), 8.20 (d, 1H), 7.03 (d, 1H).

### Step B: 4,7-Dichlorofuro[2,3-d]pyridazine

A mixture of aforementioned furo[2,3-d]pyridazine-4,7-diol (1.00 g, 6.57 mmol, 1.0 eq) in POCl₃ (0.5 mL) was degassed and purged with N₂ for three times and pyridine (1.0 mL, 12.4 mmol, 1.88 eq) was added to the mixture. The mixture was stirred at 110° C. for 3 hrs. LCMS showed desired mass was detected. The mixture was poured into ice water (100 mL) and extracted with methylene dichloride (20 mL*3). The combined organic layers were washed with brine (20 mL), dried anhydrous sodium sulfate, filtered, and the filtrate was concentrated under reduced pressure to afford the title compound (1.10 g, 88% yield) as a yellow solid. LCMS: m/z 188.9 [M+H]⁺, ESI pos.

### Step C: 2-(4-Chlorofuro[2,3-d]pyridazin-7-yl)-5-(trifluoromethyl)phenol

A mixture of aforementioned 4,7-dichlorofuro[2,3-d]pyridazine (3.00 g, 1.59 mmol, 1.0 eq), K₂CO₃ (4.38 g, 3.17 mmol, 2.0 eq) and Pd(dppf)Cl₂ (116 mg, 0.160 mmol, 0.10 eq) in 1,4-dioxane (50 mL) and water (10 mL) was degassed and purged with N₂ for three times. Then the [2-hydroxy-4-(trifluoromethyl)phenyl]boronic acid (CAS #1072951-50-8, 262 mg, 1.27 mmol, 0.80 eq) was added to the mixture. The mixture was stirred at 100° C. for 12 hrs. The mixture was concentrated under reduced pressure and purified by reversed-phase flash (CombiFlash 0.1% TFA aqueous/ACN condition) and prep-HPLC (column Waters Xbridge 150*25 mm*5 μm; Condition water (ammonia hydroxide v/v)-ACN; begin B: 35, end B: 65; gradient time (min): 9; 100% B hold time(min): 2; flow rate (mL/min): 25). After lyophilization, the title compound (110 mg, 22% yield) was obtained as yellow oil. LCMS: m/z 315.0 [M+H]⁺, ESI pos.

### Step D: 2-[4-[[(3R)-1-ethyl-3-piperidyl]amino]furo[2,3-d]pyridazin-7-yl]-5-(trifluoromethyl)phenol

To a solution of 2-(4-chlorofuro[2,3-d]pyridazin-7-yl)-5-(trifluoromethyl)phenol (28.0 mg, 0.09 mmol, 1.0 eq) and (3R)-1-ethylpiperidin-3-amine (CAS #1020396-26-2, 57.1 mg, 0.44 mmol, 5.0 eq) in 1,4-dioxane (1 mL) was added Cs₂CO₃ (72.5 mg, 0.220 mmol, 2.5 eq) and BinapPdG₃ (5.0 mg, 0.020 mmol, 0.20 eq). The mixture was stirred at 110° C. for 12 hrs under N₂. The mixture was concentrated under reduced pressure and purified by reversed-phase flash (CombiFlash 0.1% NH₃H₂O aqueous/ACN condition) and followed by lypohilization to afford the title compound (4 mg, 10% yield) as a yellow solid. LCMS: m/z 407.1 [M+H]⁺, ESI pos.

### Example 9

### 2-[7-[[(3R)-1-Ethyl-3-piperidyl]amino]-1-methyl-pyrazolo[3,4-d]pyridazin-4-yl]-5-(trifluoromethyl)phenol

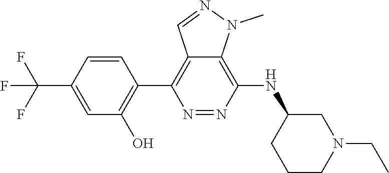 [SMILES: CCN1CCC[C@@H](Nc2nnc(-c3ccc(C(F)(F)F)cc3O)c3cnn(C)c23)C1]

### Step A: 4-Chloro-N-[(3R)-1-ethyl-3-piperidyl]-1-methyl-pyrazolo[3,4-d]pyridazin-7-amine

To a solution of 4,7-dichloro-1-methyl-pyrazolo[3,4-d]pyridazine (Example 6, step C; 200 mg, 0.99 mmol, 1.0 eq) and (3R)-1-ethylpiperidin-3-amine (CAS #1020396-26-2, 151.57 mg, 1.18 mmol, 1.2 eq) in NMP (1 mL) was added potassium carbonate (272 mg, 1.97 mmol, 2.0 eq). The mixture was stirred at 85° C. for 16 hours under N₂. Upon reaction completion, the reaction mixture was cooled to 20° C. and purified by reversed phase flash (CombiFlash, 0.1% TFA aqueous-ACN condition) to afford both regio-isomers (70.0 mg, 24% yield; INT Example 6, Step D) as yellow solid and the title compound (20.0 mg, 7% yield) as yellow solid. LCMS: m/z 295.0 [M+H]⁺, ESI pos.

### Step B: 2-[7-[[(3R)-1-Ethyl-3-piperidyl]amino]-1-methyl-pyrazolo[3,4-d]pyridazin-4-yl]-5-(trifluoromethyl)phenol

To a solution of 4-chloro-N-[(3R)-1-ethyl-3-piperidyl]-1-methyl-pyrazolo[3,4-d]pyridazin-7-amine (20 mg, 0.07 mmol, 1.0 eq) and [2-hydroxy-4-(trifluoromethyl)phenyl]boronic acid (CAS #1072951-50-8, 27.94 mg, 0.14 mmol, 2.0 eq) in 1,4-dioxane (2 mL) and water (0.40 mL) was added CsF (30.92 mg, 0.2 mmol, 3.0 eq) and Pd(dppf)Cl₂ (9.93 mg, 0.01 mmol, 0.2 eq). The above reaction mixture was stirred at 130° C. for 1.5 hour under microwave conditions. Upon reaction completion, the mixture was concentrated under reduce pressure and purified by reversed phase flash (CombiFlash, 0.1% TFA aqueous-ACN condition) to give the crude product. The crude product was purified by prep-HPLC (column: Phenomenex Synergi Polar-RP 100 mm*25 mm*4 μm; condition: water (TFA)-MeCN Begin B: 23; End B: 43; gradient Time(min): 7; 100% B Hold Time(min): 2; flowRate (ml/min): 25), to give a yellow solid, and then re-purified by prep-HPLC (column: Waters Xbridge 150*25 mm*5 μm; condition: water (NH₄HCO₃)-MeCN, Begin B: 55; End B: 85; gradient Time(min): 10; 100% B Hold Time(min): 2; flowRate (ml/min): 25) to afford the title compound (6.48 mg, 22% yield) as yellow solid. LCMS: m/z 421.1 [M+H]⁺, ESI pos.

### Example 10

### 2-[4-[[(3R)-1-Ethyl-3-piperidyl]amino]-1-methyl-pyrazolo[3,4-d]pyridazin-7-yl]-5-(trifluoromethoxy)phenol; 2,2,2-trifluoroacetic acid

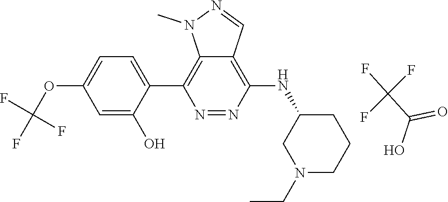

### Step A: N-[(3R)-1-ethyl-3-piperidyl]-7-[2-methoxy-4-(trifluoromethoxy)phenyl]-1-methyl-pyrazolo[3,4-d]pyridazin-4-amine

In a microwave tube, 7-chloro-N-[(3R)-1-ethyl-3-piperidyl]-1-methyl-pyrazolo[3,4-d]pyridazin-4-amine (Example 6, step D, 150.0 mg, 0.51 mmol, 1.0 eq), CsF (386.47 mg, 2.54 mmol, 5.0 eq) and 2-methoxy-4-(trifluoromethoxy)phenylboronic acid (144.08 mg, 0.61 mmol, 1.2 eq; CAS: 355836-10-1) were dissolved in 1,4-dioxane (2 mL) and water (0.4 mL) and the mixture was purged with nitrogen three times, then Pd(dppf)Cl₂ (74.39 mg, 0.1 mmol, 0.2 eq) was added to get a red solution. The reaction mixture was stirred at 130° C. for 3 hours under microwave condition to give a black solution. The reaction mixture was quenched with water (10 mL) to get a brown solution, then extracted with EtOAc (3×20 mL), washed with brine (2×30 mL), dried over anhydrous Na₂SO₄, filtered and the filtrate was concentrated under reduced pressure to give a yellow solid. The crude was purified by reversed-phase flash (CombiFlash, 0.1% TFA aqueous-ACN condition) to give the title compound (60.0 mg, 26% yield) as a yellow solid. LCMS: m/z 451.2 [M+H]+, ESI pos.

### Step B: 2-[4-[[(3R)-1-Ethyl-3-piperidyl]amino]-1-methyl-pyrazolo[3,4-d]pyridazin-7-yl]-5-(trifluoromethoxy)phenol; 2,2,2-trifluoroacetic acid

To a mixture of N-[(3R)-1-ethyl-3-piperidyl]-7-[2-methoxy-4-(trifluoromethoxy)phenyl]-1-methyl-pyrazolo[3,4-d]pyridazin-4-amine (40.0 mg, 0.09 mmol, 1.0 eq) in DCM (0.5 mL) was added BBr₃ (222.46 mg, 0.89 mmol, 10.0 eq) under N₂ and stirred at −60° C. for 10 minutes, then stirred at 25° C. for 1 hour. The reaction mixture was quenched by addition of ice water (2 mL) and neutralized with NH₃—H₂O solution, filtered and the filtrate was concentrated under reduced pressure to get a yellow solid. The crude was purified by reversed-phase flash (CombiFlash, 0.1% TFA aqueous-ACN condition) to give the title compound (29.7 mg, 58% yield) as a white solid. LCMS: m/z 437.2 [M+H]⁺, ESI pos.

### Example 11

### 2-[4-[[(1R,2R)-2-Hydroxycyclohexyl]amino]-1-methyl-pyrazolo[3,4-d]pyridazin-7-yl]-5-(trifluoromethyl)phenol; 2,2,2-trifluoroacetic acid

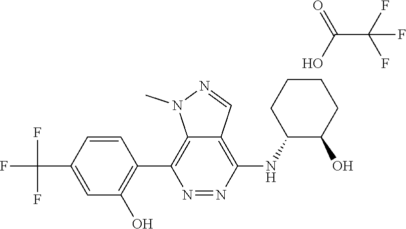

### Step A: (1R,2R)-2-((7-chloro-1-methyl-1H-pyrazolo[3,4-d]pyridazin-4-yl)amino)cyclohexan-1-ol and (1R,2R)-2-((4-chloro-1-methyl-1H-pyrazolo[3,4-d]pyridazin-7-yl)amino)cyclohexan-1-ol

To a solution of (1R,2R)-2-aminocyclohexanol hydrochloride (CAS: 13374-31-7, 896.2 mg, 5.91 mmol, 8.0 eq) in NMP (5 mL) was added K₂CO₃ (815.64 mg, 5.91 mmol, 8.0 eq) and 4,7-dichloro-1-methyl-pyrazolo[3,4-d]pyridazine (Example 6, step C; 150.0 mg, 0.74 mmol, 1.0 eq) under N₂, and then the mixture was stirred at 115° C. for 16 hours. The reaction mixture was quenched with water (2 mL) and then concentrated in vacuum. The residue was purified by reversed-phase flash (CombiFlash, 0.1% TFA aqueous-ACN condition) to give the desired product (1R,2R)-2-[(7-chloro-1-methyl-pyrazolo[3,4-d]pyridazin-4-yl)amino]cyclohexanol (45.0 mg, 14% yield) as a light yellow solid, LCMS: m/z 282.2 [M+H]⁺, ESI pos. As a side product, (1R,2R)-2-[(4-chloro-1-methyl-pyrazolo[3,4-d]pyridazin-7-yl)amino]cyclohexanol (40.0 mg, 19% yield) was isolated as a light yellow solid, LCMS: m/z 282.2 [M+H]⁺, ESI pos.

### Step B: 2-[4-[[(1R,2R)-2-Hydroxycyclohexyl]amino]-1-methyl-pyrazolo[3,4-d]pyridazin-7-yl]-5-(trifluoromethyl)phenol; 2,2,2-trifluoroacetic acid

To a solution of [2-hydroxy-4-(trifluoromethyl)phenyl]boronic acid (CAS #1072951-50-8, 43.86 mg, 0.210 mmol, 1.5 eq) (1R,2R)-2-[(7-chloro-1-methyl-pyrazolo[3,4-d]pyridazin-4-yl)amino]cyclohexanol (40.0 mg, 0.140 mmol, 1 eq), CsF (86.26 mg, 0.570 mmol, 4 eq) in 1,4-dioxane (1 mL)/water (0.2 mL) was added Pd(dppf)Cl₂ (5.19 mg, 0.01 mmol, 0.05 eq) under N₂ at 25° C. The reaction mixture was heated under microwave irradiation at 125° C. for 2 hours. Upon reaction completion, the mixture was concentrated under reduced pressure and purified by reversed-phasee flash (CombiFlash, 0.1% TFA aqueous-ACN condition) to afford the title compound (29.0 mg, 38% yield) as a white solid. LCMS: m/z 408.0 [M+H]⁺, ESI pos.

### Example 12

### 5-Chloro-2-[1-methyl-4-[[(3R)-1-ethyl-3-piperidyl]amino]pyrazolo[3,4-d]pyridazin-7-yl]phenol; 2,2,2-trifluoroacetic acid

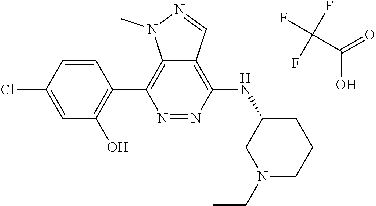 [SMILES: CCN1CCC[C@@H](Nc2nnc(-c3ccc(Cl)cc3O)c3c2cnn3C)C1 ; O=C(O)C(F)(F)F]

### Step A: 4-Chloro-N-[(3R)-1-ethyl-3-piperidyl]-1-methyl-pyrazolo[3,4-d]pyridazin-7-amine

To a mixture of 7-chloro-N-[(3R)-1-ethyl-3-piperidyl]-1-methyl-pyrazolo[3,4-d]pyridazin-4-amine (Example 6, step D; 80.0 mg, 0.27 mmol, 1.0 eq.), 4-chloro-2-hydroxyphenyl boronic acid (CAS: 1238196-66-1, 93.6 mg, 0.54 mmol, 2.0 eq) and Cs₂CO₃ (265.26 mg, 0.81 mmol, 3.0 eq) in 1,4-dioxane (1.5 mL) and Water (0.3 mL) was added XPhos Pd G₃ (34.5 mg, 0.04 mmol, 0.15 eq.) at 25° C., and the mixture was stirred at 100° C. for 4 hour under N2 to get a brown solution. The reaction mixture was quenched by 2 mL water, then adjust pH<5 by addition of 2 mL 1N HCl solution, and then diluted with 2 mL MeOH to get a brown solution which was purified by reversed-phase flash (CombiFlash 0.1% TFA aqueous-ACN condition) and followed by lyophilization to give the title compound (22.0 mg, 15% yield) as a white solid. LCMS: m/z 387.2 [M+H]⁺, ESI pos.

### Example 13

### 2-[4-[[(3R)-1-Ethyl-3-piperidyl]amino]-3-methyl-isoxazolo[4,5-d]pyridazin-7-yl]-5-(trifluoromethyl)phenol

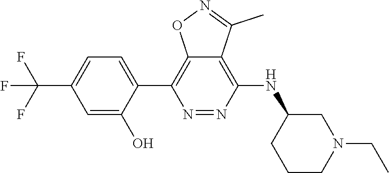 [SMILES: CCN1CCC[C@@H](Nc2nnc(-c3ccc(C(F)(F)F)cc3O)c3onc(C)c23)C1]

### Step A: 7-Chloro-N-[(3R)-1-ethyl-3-piperidyl]-3-methyl-isoxazolo[4,5-d]pyridazin-4-amine

4,7-Dichloro-3-methyl-isoxazolo[4,5-d]pyridazine (124.0 mg, 0.61 mmol, 1.0 eq, CAS #106584-70-7), DIPEA (0.53 mL, 3.04 mmol, 5.0 eq) and [(3R)-1-ethylpiperidin-1-ium-3-yl]ammonium; dichloride (128.37 mg, 0.64 mmol, 1.05 eq) were dissolved in NMP (5 mL) and stirred at 110° C. for 16 h. The reaction mixture was diluted with MeOH (20 mL) and stirred with SCX (6 g) for 30 min. The mixture was filtered, and the resin washed with MeOH (20 mL). The crude product was then eluted with 0.7 N NH₃ in MeOH (50 mL) to afford a brown oil. The crude product was purified by chromatography on silica gel (0-10% (0.7 N NH₃ in MeOH)/DCM) to afford the title compound (83.0 mg, 42% yield) as a yellow oil. ¹H NMR (500 MHz, DMSO-d₆) δ [ppm]: 6.48 (d, 1H), 4.39-4.26 (m, 1H), 2.96-2.84 (m, 1H), 2.74-2.53 (m, 4H), 2.37 (q, 2H), 2.22-2.13 (m, 2H), 1.89-1.81 (m, 1H), 1.77-1.68 (m, 1H), 1.65-1.47 (m, 2H), 1.01 (t, 3H).

### Step B: 2-[4-[[(3R)-1-Ethyl-3-piperidyl]amino]-3-methyl-isoxazolo[4,5-d]pyridazin-7-yl]-5-(trifluoromethyl)phenol

Aforementioned 7-chloro-N-[(3R)-1-ethyl-3-piperidyl]-3-methyl-isoxazolo[4,5-d]pyridazin-4-amine (90.0 mg, 0.3 mmol, 1.0 eq), caesium fluoride (596.41 mg, 1.22 mmol, 4.0 eq), XPhos Pd G3 (25.79 mg, 0.03 mmol, 0.1 eq; CAS #1445085-55-1) and 2-methoxy-4-(trifluoromethyl)-phenylboronic acid (93.7 mg, 0.43 mmol, 1.4 eq; CAS #312936-89-3) were suspended in DMF (2 mL) and the vessel evacuated and back-filled with N₂ (3 x). The reaction mixture was stirred at 110° C. for 24 h. The reaction mixture was cooled, diluted with EtOAc (20 mL), washed with brine (20 mL) and 10 wt % aqueous LiCl (20 mL), then dried using a phase separator and concentrated in vacuo. The resulting residue was dissolved in DCM (3 mL) and BBr₃ (1.0 M in DCM, 0.91 mL, 0.91 mmol, 3.0 eq) was added. The reaction mixture was stirred at r.t. for 1 h, then concentrated in vacuo. The resulting residue was taken up in DCM (10 mL) and NaHCO₃ (1 g) was added. The reaction mixture was stirred for 20 min, then filtered and the filtrate was concentrated in vacuo. The resulting residue was dissolved in a mixture of DMSO/MeOH/DCM, filtered and purified by reversed phase preparative HPLC (Waters 2767 Sample Manager, Waters 2545 Binary Gradient Module, Waters Systems Fluidics Organiser, Waters 515 ACD pump, Waters 515 Makeup pump, Waters 2998 Photodiode Array Detector, Waters QDa) on a Waters XBridge BEH C18 ODB prep column, 130 Å, 5 μm, 30 mm×100 mm, flow rate 40 mL min-1 eluting with a 0.3% Ammonia in water-MeCN gradient over 12.5 mins using UV across all wavelengths with PDA as well as a QDA and ELS detector. At-column dilution pump gives 2 mL min-1 Methanol over the entire method, which is included in the following MeCN percentages. Gradient information: 0.0-0.5 min, 55% MeCN; 0.5-10.5 min, ramped from 55% MeCN to 85% MeCN; 10.5-10.6 min, ramped from 85% MeCN to 100% MeCN; 10.6-12.5 min, held at 100% MeCN. This afforded the title compound (4.3 mg, 3% yield) as a light brown solid. LCMS m/z: 421.8 (M+H)⁺, ESI pos.

### Example 14

### 2-[4-[[(3R)-1-ethyl-3-piperidyl]amino]-1H-pyrazolo[3,4-d]pyridazin-7-yl]-3-methyl-5-(trifluoromethyl)phenol; 2,2,2-trifluoroacetic acid

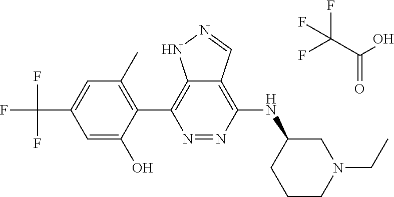 [SMILES: CCN1CCC[C@@H](Nc2nnc(-c3c(C)cc(C(F)(F)F)cc3O)c3[nH]ncc23)C1 ; O=C(O)C(F)(F)F]

### Step A: 1H-Pyrazole-4,5-dicarbohydrazide

To a solution of diethyl 1H-pyrazole-4,5-dicarboxylate (CAS: 37687-26-6, 1.0 g, 4.71 mmol, 1.0 eq) in ethanol (20 mL) was added hydrazine monohydrate (1.6 mL, 33 mmol, 7.0 eq) at 20° C.

The mixture was slowly heated to 70° C. and stirring was continued at 70° C. for 5 hours. Then, the mixture was cooled to 20° C., and the suspension was filtered and the filter cake was washed with EtOH (3×20 mL) and dried under vacuum to give the title compound (800.0 mg, 92% yield) as a white solid.

### Step B: 1H-Pyrazolo[3,4-d]pyridazine-4,7-diol

To a solution of 2-methylpyrazole-3,4-dicarbohydrazide (0.8 g, 4.04 mmol, 1.0 eq.) in water (4 mL) was added HCl (2.29 mL, 27.43 mmol, 6.79 eq) at 20° C. dropwise. The mixture was stirred at 100° C. for 2 hours. After cooling to ambient temperature, the suspension was diluted with water (20 mL) and filtered. The filter cake was washed with EtOH (3×10 mL) and dried in vacuum to give the title compound (570.0 mg, 85% yield) as a white solid.

### Step C: 4,7-Dichloro-1H-pyrazolo[3,4-d]pyridazine

A mixture of 1H-pyrazolo[3,4-d]pyridazine-4,7-diol (0.92 g, 6.02 mmol, 1.0 eq) was added to POCl₃ (10.0 mL). The mixture was stirred at 60° C. for 12 hours. Then, the mixture was concentrated in under vacuum to remove the excess POCl₃ and to give crude title compound (600.0 mg, 53% yield) which was used directly in the next step.

### Step D: 4,7-Dichloro-1-((2-(trimethylsilyl)ethoxy)methyl)-1H-pyrazolo[3,4-d]pyridazin

To a solution of 4,7-dichloro-1H-pyrazolo[3,4-d]pyridazine (0.22 g, 1.14 mmol, 1.0 eq) in THF (5 mL) was added NaH (68.57 mg, 1.71 mmol, 1.5 eq) at 0° C. in portion and then stirred at 0° C. for 0.25 h, and then was added SEMCl (286.29 mg, 1.71 mmol, 1.5 eq) at 0° C. for 2 h. Upon reaction completion, the mixture was quenched with H₂O (30 mL), and extracted with EtOAc (30 mL×3). The organic phase was washed with brine (30 mL×2), dried over anhydrous Na₂SO₄, filtered and concentrated in vacuum. The residue was purified over column chromatography (hexane/EtOAc, 5:1) to afford the title compound (85.0 mg, 23% yield) as light yellow oil. LCMS: m/z 319.2 [M+H]⁺, ESI pos.

### Step E: (R)-7-chloro-N-(1-ethylpiperidin-3-yl)-1-((2-(trimethylsilyl)ethoxy)methyl)-1H-pyrazolo[3,4-d]pyridazin-4-amine

To a solution of 4,7-dichloro-1-((2-(trimethylsilyl)ethoxy)methyl)-1H-pyrazolo[3,4-d]pyridazine (65.0 mg, 0.2 mmol, 1.0 eq) and (3R)-1-ethylpiperidin-3-amine (33.94 mg, 0.26 mmol, 1.3 eq) in NMP (2 mL) was added DIEA (78.91 mg, 0.61 mmol, 3.0 eq). The mixture was stirred at 85° C. for 16 h under N2. Upon reaction completion, the mixture was concentrated under reduced pressure and purified by reversed-phase flash (CombiFlash, 0.1% TFA aqueous-ACN condition) to afford the title compound (10.0 mg, 12% yield) as a yellow oil. LCMS: m/z 411.2 [M+H]⁺, ESI pos.

### Step F: 2-(4-(((R)-1-ethylpiperidin-3-yl)amino)-1-((2-(trimethylsilyl)ethoxy)methyl)-1H-pyrazolo[3,4-d]pyridazin-7-yl)-3-methyl-5-(trifluoromethyl)phenol

To a solution of (R)-7-chloro-N-(1-ethylpiperidin-3-yl)-1-((2-(trimethylsilyl)ethoxy)methyl)-1H-pyrazolo[3,4-d]pyridazin-4-amine (15.0 mg, 0.04 mmol, 1.0 eq.) and [2-hydroxy-6-methyl-4-(trifluoromethyl)phenyl]boronic acid (32.11 mg, 0.15 mmol, 4.0 eq) in 1,4-dioxane (1 mL) and water (0.2 mL) was added CsF (22.17 mg, 0.15 mmol, 4.0 eq) and Xphos Pd G₃ (3.09 mg, 0.1 eq) under N₂. The mixture was stirred at 95° C. for 5 hours. Upon reaction completion, the mixture was concentrated under reduced pressure, and the residue was purified by preparative TLC (DCM/MeOH 10:1) to give the title compound (9.0 mg, 67% yield) as a yellow oil. LCMS: m/z 551.3 [M+H]⁺, ESI pos.

### Step G: 2-[4-[[(3R)-1-ethyl-3-piperidyl]amino]-1H-pyrazolo[3,4-d]pyridazin-7-yl]-3-methyl-5-(trifluoromethyl)phenol; 2,2,2-trifluoroacetic acid

To a solution of 2-[4-[[(3R)-1-ethyl-3-piperidyl]amino]-1-(2-tri-methylsilylethoxymethyl)-pyrazolo[3,4-d]pyridazin-7-yl]-3-methyl-5-(trifluoromethyl)phenol (9.0 mg, 0.02 mmol, 1.0 eq) in DCM (0.5 mL) was added TFA (0.5 mL). The mixture was stirred at 25° C. for 2 hours. Upon reaction completion, the mixture was concentrated under reduce pressure, and the residue was purified by prep-HPLC (column: Phenomenex Synergi Polar-RP 100*25 mm*4 μm; condition: water (TFA)-ACN; Begin B: 12; End B: 32; Gradient Time(min): 7; FlowRate (ml/min): 25) to give the desired product (1.58 mg, 18% yield) as a yellow solid. LCMS: m/z 421.2 [M+H]⁺, ESI pos.

### Example 15

### 2-[4-[[(3R)-1-Ethyl-3-piperidyl]amino]-1-methyl-pyrazolo[3,4-d]pyridazin-7-yl]-3-fluoro-5-(trifluoromethyl)phenol

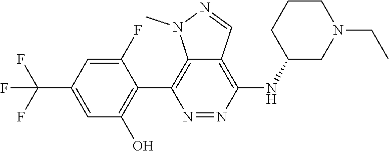 [SMILES: CCN1CCC[C@@H](Nc2nnc(-c3c(O)cc(C(F)(F)F)cc3F)c3c2cnn3C)C1]

### Step A: 2-Bromo-6-fluoro-4-(trifluoromethyl)aniline

To a solution of commercially available 2-fluoro-4-(trifluoromethyl)aniline (25.0 g, 140 mmol, 1.00 eq) in DMF (300 mL) was added NBS (26.1 g, 147 mmol, 1.05 eq) at −10° C. The mixture was stirred at 25° C. for 12 h. The reaction mixture was diluted with EtOAc (500 mL) and extracted. The organic phase was washed with brine (500 mL*3), dried over Na₂SO₄, filtered and concentrated in vacuum. The residue was purified by column chromatography (SiO₂, Petroleum ether/Ethyl acetate=1/0 to 10/1) to afford the title compound (36.0 g, 99.9% yield) as yellow oil. LCMS: m/z 257.9 [M+H]⁺, ESI pos.

### Step B: 2-Fluoro-6-(4,4,5,5-tetramethyl-1,3,2-dioxaborolan-2-yl)-4-(trifluoromethyl)aniline

To a solution of compound 2-bromo-6-fluoro-4-(trifluoromethyl)aniline (30.0 g, 116 mmol, 1.00 eq) in dioxane (500 mL) was added 4,4,5,5-tetramethyl-2-(4,4,5,5-tetramethyl-1,3,2-dioxaborolan-2-yl)-1,3,2-dioxaborolane (59.1 g, 233 mmol, 2.00 eq), KOAc (28.5 g, 291 mmol, 2.50 eq) and Pd(dppf)Cl₂·CH₂Cl₂ (9.50 g, 11.6 mmol, 0.10 eq) under N₂. The mixture was stirred at 100° C. for 3 h. The reaction was concentrated in vacuum. The residue diluted with EtOAc (1000 mL) and extracted. The organic phase was washed with brine (1000 mL), dried over Na₂SO₄, filtered and concentrated in vacuum to afford the title compound (45.0 g) as black oil, which was used directly in next step. LCMS: m/z 306.1 [M+H]⁺, ESI pos.

### Step C: 2-Amino-3-fluoro-5-(trifluoromethyl)phenol

To a solution of aforementioned 2-fluoro-6-(4,4,5,5-tetramethyl-1,3,2-dioxaborolan-2-yl)-4-(trifluoromethyl)aniline (45.0 g, 148 mmol, 1.00 eq) in THF (600 mL) was added NaOH (2M, 221 mL, 3.00 eq) and H₂O₂ (100 g, 885 mmol, 85.0 mL, 30.0% purity, 6.00 eq) at 0° C. and the reaction was stirred for 3 hrs at 25° C. The reaction was diluted with EtOAc (1500 mL) and extracted. The organic phase was washed with aqueous Na₂SO₃ solution (1500 mL*3), dried over Na₂SO₄, filtered and concentrated under reduced pressure to give a residue. The crude product was purified by reversed-phase HPLC (0.1% formic acid condition) to afford the title compound (11.0 g, 38% yield) as a brown solid. LCMS: m/z 196.0 [M+H]⁺, ESI pos.

### Step D: 3-Fluoro-2-iodo-5-(trifluoromethyl)phenol

To a solution of compound 2-amino-3-fluoro-5-(trifluoromethyl)phenol (11.0 g, 56.4 mmol, 1.00 eq) and H₂SO₄ (40.5 g, 404 mmol, 22.0 mL, 7.17 eq) in H₂O (200 mL) and acetone (50.0 mL) was added NaNO₂ (7.78 g, 113 mmol, 2.00 eq) at 0° C. and the reaction was stirred for 30 min at 0° C. Then CuI (26.8 g, 141 mmol, 2.50 eq) and NaI (21.1 g, 141 mmol, 2.50 eq) were added to the reaction at 0° C. and the reaction was stirred for 1.5 h at 0° C. After reaction completion, water (500 mL) was added to the reaction mixture. The water phase was washed with EtOAc (300 mL*2). The combined organic layers were washed with brine (300 mL*2), dried over Na₂SO₄, filtered and concentrated under reduced pressure to give a residue. The residue was purified by column chromatography (SiO₂, Petroleum ether/Ethyl acetate=1/0 to 10/1) to afford the title compound (20.0 g) as brown oil. ¹H NMR (400 MHz, CDCl₃) δ=7.04 (s, 1H), 6.89 (dd, 1H), 6.76 (s, 1H).

### Step E: 1-(Ethoxymethoxy)-3-fluoro-2-iodo-5-(trifluoromethyl)benzene

To a solution of compound 3-fluoro-2-iodo-5-(trifluoromethyl)phenol (20.0 g, 65.4 mmol, 1.00 eq) and chloromethoxyethane (9.27 g, 98.0 mmol, 9.09 mL, 1.50 eq) in DMF (200 mL) was added Cs₂CO₃ (31.9 g, 98.0 mmol, 1.50 eq) and the mixture was stirred at 25° C. for 2 h. After reaction cpmpletion, EtOAc (500 mL) was added and the phase were separated and extracted. The organic phase was washed with brine (500 mL*3), dried over Na₂SO₄, filtered and concentrated under reduced pressure to give a residue which was purified by column chromatography (SiO₂, Petroleum ether/Ethyl acetate=1/0 to 10/1) to afford the title compound (10.0 g, 42% yield) as colorless oil. ¹H NMR (400 MHz, CDCl₃): δ=7.15 (s, 1H), 7.00 (dd, 1H), 5.36 (s, 2H), 3.78 (q, 2H), 1.24 (t, 3H).

### Step F: 2-[2-(Ethoxymethoxy)-6-fluoro-4-(trifluoromethyl)phenyl]-4,4,5,5-tetramethyl-1,3,2-dioxaborolane

To a solution of 1-(ethoxymethoxy)-3-fluoro-2-iodo-5-(trifluoromethyl)benzene (10.0 g, 27.5 mmol, 1.00 eq) and 2-isopropoxy-4,4,5,5-tetramethyl-1,3,2-dioxaborolane (15.3 g, 82.4 mmol, 16.8 mL, 3.00 eq) in THF (100 mL) was added n-BuLi (2.50 M, 27.5 mL, 2.50 eq) at −70° C. and the reaction was stirred for 1 h at −70° C. After reaction completion, was aq. NH₄Cl solution (300 mL) added and the mixture was stirred for 10 min, extracted with EtOAc (200 mL*2). The combined organic layers were washed with brine (300 mL), dried over Na₂SO₄, filtered and concentrated under reduced pressure to give a residue. The residue was purified by prep-HPLC (Column: Welch Ultimate XB-CN 250*50*10 μm; mobile phase: [Hexane-EtOH]; B %: 0%-0%, 7 min) to afford the title compound (7.00 g, 60% yield, 86.3% purity) as a white solid. ¹H NMR (400 MHz, CDCl₃): δ=7.10 (s, 1H), 6.94 (d, 1H), 5.24 (s, 2H), 3.73 (q, 2H), 1.39 (s, 12H), 1.22 (t, 3H).

### Step G: 7-[2-(Ethoxymethoxy)-6-fluoro-4-(trifluoromethyl)phenyl]-1-methyl-N-[(3R)-1-ethyl-3-piperidyl]pyrazolo[3,4-d]pyridazin-4-amine

A mixture of 7-chloro-N-[(3R)-1-ethyl-3-piperidyl]-1-methyl-pyrazolo[3,4-d]pyridazin-4-amine (70 mg, 0.24 mmol, 1.0 eq; Example 6, step D), aforementioned 2-[2-(ethoxymethoxy)-6-fluoro-4-(trifluoromethyl)phenyl]-4,4,5,5-tetramethyl-1,3,2-dioxaborolane (201.5 mg, 0.48 mmol, 2.0 eq), potassium carbonate (131.3 mg, 0.95 mmol, 4.0 eq) and SPhos Pd G3 (25.1 mg, 0.03 mmol, 0.14 eq) in 1,4-dioxane (2.81 mL) and water (0.7 mL) was flushed with argon and stirred at 110° C. for 3 h. An additional equivalent of boronic acid and 0.1 equivalents of SPhos Pd G3 were added and the reaction stirred at 90° C. overnight. The reaction mixture was cooled to r.t. and extracted with ethyl acetate (˜30 mL) and half-saturated aq. NH₄Cl-solution (˜0.5 mL). The aqueous layer was backextracted with ethyl acetate. The organic layers were washed with water and brine. The combined organic layers were dried over sodium sulfate, filtered and concentrated in vacuo. The crude was used without further purification in the next step. LCMS m/z: 439.3; 497.3 [M+H]⁺, ESI pos.

### Step H: 2-[4-[[(3R)-1-Ethyl-3-piperidyl]amino]-1-methyl-pyrazolo[3,4-d]pyridazin-7-yl]-3-fluoro-5-(trifluoromethyl)phenol

To a solution of 7-[2-(ethoxymethoxy)-6-fluoro-4-(trifluoromethyl)phenyl]-1-methyl-N-[(3R)-1-ethyl-3-piperidyl]pyrazolo[3,4-d]pyridazin-4-amine (100 mg, 0.20 mmol, 1.0 eq) and dichloromethane (4.1 mL) was added under ice cooling TFA (689 mg, 466 μL, 6.0 mmol, 30 eq) dropwise. The reaction mixture was stirred at 0° and then allowed to come to r.t. while stirring was continued for 2 hours. Then, the mixture was concentrated under reduced pressure and the crude was dissolved in DCM, washed with sat. aq. NaHCO₃ solution and the organic phase was separated and washed once more with water and brine. The aqueous phases were back-extracted with DCM. The combined organic layers were dried over sodium sulfate, filtered, and concentrated in vacuo. The residue was purified by flash chromatography on silica gel (gradient 0% to 100% (dichloromethane:methanol:NH₄OH; 110:10:1) in dichloromethane) to afford the title compound (50 mg, 57% yield). LCMS m/z: 439.2 [M+H]⁺, ESI pos.

### Example 16

### 2-[4-[(3-Hydroxy-3-methyl-cyclobutyl)amino]-1-methyl-pyrazolo[3,4-d]pyridazin-7-yl]-5-(trifluoromethyl)phenol

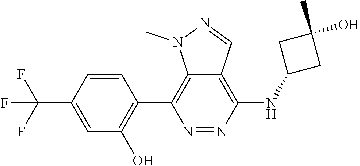 [SMILES: Cn1ncc2c(N[C@H]3C[C@@](C)(O)C3)nnc(-c3ccc(C(F)(F)F)cc3O)c21]

### Step A: 3-[(7-Chloro-1-methyl-pyrazolo[3,4-d]pyridazin-4-yl)amino]-1-methyl-cyclobutanol

To a mixture of 4,7-dichloro-1-methyl-pyrazolo[3,4-d]pyridazine (Example 6, step C) (130 mg, 0.61 mmol, 1.00 eq) in 1,4-dioxane (1.0 mL) and water (0.10 mL) was added N,N-diisopropylethylamine (337 mg, 0.455 mL, 2.61 mmol, 4.28 eq) and cis-3-amino-1-methylcyclobutan-1-ol hydrochloride (CAS #1523606-23-6, 125 mg, 0.91 mmol, 1.49 eq). The mixture was stirred at 100° C. for 16 hours. The reaction mixture was cooled to room temperature and then extracted with ethyl acetate and water. The aqueous layer was backextracted with ethyl acetate. The organic layers were washed with water and brine. The combined organic layers were dried over sodium sulfate, filtered and concentrated in vacuo. The crude product was adsorbed on ISOLUTE HM-N and purified by flash chromatography (silica gel, 12 g, gradient 0% to 5% methanol in dichloromethane). All fractions containing product were combined and concentrated to afford the title compound (57 mg, 33% yield) as an off-white solid. LCMS: m/z 268.1 [M+H]⁺, ESI pos.

### Step B: 2-[4-[(3-Hydroxy-3-methyl-cyclobutyl)amino]-1-methyl-pyrazolo[3,4-d]pyridazin-7-yl]-5-(trifluoromethyl)phenol

A mixture of 3-[(7-chloro-1-methyl-pyrazolo[3,4-d]pyridazin-4-yl)amino]-1-methyl-cyclobutanol (Example 16, step A) (54 mg, 0.19 mmol, 1.00 eq), [2-hydroxy-4-(trifluoromethyl)phenyl]boronic acid (CAS #1072951-50-8, 80 mg, 0.39 mmol, 2.03 eq), potassium carbonate (130 mg, 0.94 mmol, 4.91 eq) and 1,1′-bis(diphenylphosphino)ferrocene-palladium(II) dichloride dichloromethane complex (20 mg, 0.02 mmol, 0.13 eq) in 1,4-dioxane (1.1 mL) and water (0.55 mL) was flushed with argon and stirred at 100° C. for 16 hours. The reaction mixture was cooled to room temperature and then extracted with ethyl acetate and half-saturated aq. NH₄Cl-solution. The aqueous layer was backextracted with ethyl acetate. The organic layers were washed with water and brine. The combined organic layers were dried over sodium sulfate, filtered and concentrated in vacuo. The crude product was adsorbed on ISOLUTE HM-N and purified by flash chromatography (silica gel, 12 g, gradient 0% to 20% methanol in dichloromethane). All fractions containing product were combined and concentrated in vacuo. The residue was triturated with ethyl acetate to afford the title compound (25 mg, 32% yield) as dark brown powder. LCMS: m/z 394.3 [M+H]⁺, ESI pos.

### Example 17

### 5-[4-[[(3R)-1-Ethyl-3-piperidyl]amino]-1-methyl-pyrazolo[3,4-d]pyridazin-7-yl]-2,3-dihydrobenzofuran-4-ol

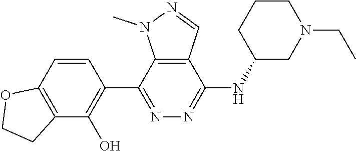 [SMILES: CCN1CCC[C@@H](Nc2nnc(-c3ccc4c(c3O)CCO4)c3c2cnn3C)C1]

### Step A: 4-Benzyloxy-5-bromo-2,3-dihydrobenzofuran

To a solution of 5-bromocoumaran-4-ol (CAS #2279149-27-6, 4.59 g, 20.26 mmol, 1.00 eq) in acetonitrile (40 mL) was added potassium carbonate (5.6 g, 40.51 mmol, 2.00 eq) followed by benzyl bromide (4.89 g, 3.4 mL, 28.57 mmol, 1.41 eq). The reaction mixture was stirred at room temperature for 2 hours. The reaction mixture was extracted with ethyl acetate and water. The aqueous layer was backextracted with ethyl acetate. The organic layers were washed with water and brine. The combined organic layers were dried over sodium sulfate, filtered and concentrated in vacuo. The crude product was adsorbed on ISOLUTE HM-N and purified by flash chromatography (silica gel, 220 g, gradient 0% to 10% ethyl acetate in heptane) to afford the title compound (6.17 g, 95% yield) as a colorless oil. LCMS: m/z 305.1/307.0 [M+H]⁺, ESI pos.

### Step B: 2-(4-Benzyloxy-2,3-dihydrobenzofuran-5-yl)-4,4,5,5-tetramethyl-1,3,2-dioxaborolane

To a solution of 4-benzyloxy-5-bromo-2,3-dihydrobenzofuran (Example 17, step A) (6.16 g, 19.18 mmol, 1.00 eq) and 2-isopropoxy-4,4,5,5-tetramethyl-1,3,2-dioxaborolane (CAS #61676-62-8, 5.47 g, 6.0 mL, 29.41 mmol, 1.53 eq) in tetrahydrofuran (80 mL) was added dropwise n-butyllithium, 1.6 M solution in hexanes (19 mL, 30.4 mmol, 1.59 eq) within 40 minutes at −76° C. Let stir at −76° C. for 2.5 hours. The reaction mixture was warmed to −60° C., quenched with saturated aq. NH₄Cl-solution at −60° C., warmed to room temperature and then extracted with ethyl acetate and saturated aq. NH₄Cl-solution. The aqueous layer was backextracted with ethyl acetate. The organic layers were washed with brine. The combined organic layers were dried over sodium sulfate, filtered and concentrated in vacuo. The crude product was adsorbed on ISOLUTE HM-N and purified by flash chromatography (silica gel, 120 g, gradient 0% to 10% ethyl acetate in heptane) to afford the title compound (5.78 g, 81% yield) as a colorless oil. LCMS: m/z 353.1 [M+H]⁺, ESI pos.

### Step C: 5-(4,4,5,5-Tetramethyl-1,3,2-dioxaborolan-2-yl)-2,3-dihydrobenzofuran-4-ol

A solution of 2-(4-benzoxycoumaran-5-yl)-4,4,5,5-tetramethyl-1,3,2-dioxaborolane (Example 17, step B) (5.77 g, 15.56 mmol, 1.00 eq) in ethyl acetate (70 mL) was three times alternating evacuated and flushed with argon. Palladium on activated charcoal, 10% Pd basis (577 mg, 0.54 mmol, 0.03 eq) was added. The reaction flask was evacuated, flushed with argon, evacuated and flushed with hydrogen. The reaction mixture was stirred under hydrogen atmosphere (balloon) at room temperature for 3 hours. Methanol (10 mL) was added. The reaction flask was three times alternating evacuated and flushed with argon, evacuated and then flushed with hydrogen. The reaction mixture was stirred under hydrogen atmosphere (balloon) at room temperature for 1 hour. The reaction mixture was filtered and rinsed well with ethyl acetate/methanol. The filtrate was concentrated in vacuo to afford the title compound (4.22 g, 98% yield) as an off-white solid, which was used without further purification. LCMS: m/z 263.2 [M+H]⁺, ESI pos.

### Step D: 5-[4-[[(3R)-1-Ethyl-3-piperidyl]amino]-1-methyl-pyrazolo[3,4-d]pyridazin-7-yl]-2,3-dihydrobenzofuran-4-ol

A mixture of 7-chloro-N-[(3R)-1-ethyl-3-piperidyl]-1-methyl-pyrazolo[3,4-d]pyridazin-4-amine (Example 6, step D) (300 mg, 1.02 mmol, 1.00 eq), 5-(4,4,5,5-tetramethyl-1,3,2-dioxaborolan-2-yl)-2,3-dihydrobenzofuran-4-ol (Example 17, step C) (570 mg, 1.87 mmol, 1.84 eq), potassium carbonate (675 mg, 4.88 mmol, 4.80 eq) and 1,1′-bis(diphenylphosphino)ferrocene-palladium(II) dichloride dichloromethane complex (126 mg, 0.15 mmol, 0.15 eq) in 1,4-dioxane (6.0 mL) and water (3.0 mL) was flushed with argon and stirred at 95° C. for 16 hours. The reaction mixture was cooled to room temperature and then extracted with ethyl acetate and half-saturated aq. NH₄Cl-solution. The aqueous layer was backextracted twice with ethyl acetate. The organic layers were washed with water and brine. The combined organic layers were dried over sodium sulfate, filtered and concentrated in vacuo.

The crude product was adsorbed on ISOLUTE HM-N and purified by flash chromatography (silica gel, 25 g, gradient 0% to 100% (dichloromethane:methanol:NH₄OH 9:1:0.05) in dichloromethane). All fractions containing product were combined and concentrated in vacuo. The residue was adsorbed on ISOLUTE HM-N and repurified by flash chromatography (Si-amine, 25 g, gradient 0% to 10% methanol in ethyl acetate). All fractions containing product were combined and concentrated in vacuo. The residue was adsorbed on ISOLUTE HM-N and repurified by flash chromatography (silica gel, 25 g, gradient 0% to 20% methanol in dichloromethane). All fractions containing product were combined and concentrated in vacuo to afford the title compound (234 mg, 57% yield) as a yellow foam. LCMS: m/z 395.3 [M+H]⁺, ESI pos.

### Example 18

### 3-[4-[[(3R)-1-Ethyl-3-piperidyl]amino]-1-methyl-pyrazolo[3,4-d]pyridazin-7-yl]bicyclo[4.2.0]octa-1(6),2,4-trien-2-ol

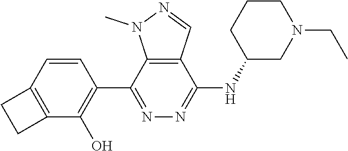 [SMILES: CCN1CCC[C@@H](Nc2nnc(-c3ccc4c(c3O)CC4)c3c2cnn3C)C1]

### Step A: 2-[(3-Bromo-2-bicyclo[4.2.0]octa-1,3,5-trienyl)oxymethoxy]ethyl-trimethyl-silane

To a solution of 3-bromobicyclo[4.2.0]octa-1,3,5-trien-2-ol (WO2021150574, 195 mg, 0.98 mmol, 1.0 eq) in DMF (5 mL) was added potassium carbonate (302 mg, 2.19 mmol, 2.20 eq) at room temperature. The resulting mixture was sonicated then 2-(trimethylsilyl)ethoxymethyl chloride (200 μL, 1.13 mmol, 1.15 eq) was added and the reaction mixture was stirred at room temperature for 16 h. Then potassium carbonate (140 mg, 1.01 mmol, 1.03 eq) followed by 2-(trimethylsilyl)ethoxymethyl chloride (0.1 mL, 0.570 mmol, 0.58 eq) was added and the reaction mixture was stirred at room temperature for 2 h. The reaction mixture was diluted with EtOAc (50 mL) and 50 v % brine (100 mL) and the separated aqueous layer was further extracted with EtOAc (2×50 mL). The combined organic layers were washed with 50 v % brine (100 mL), dried (MgSO4), filtered and concentrated. The crude reaction mixture was purified by column chromatography on silica gel (40 g, 0-20% MTBE:isoHexane) to afford the title compound (345.0 mg, 100% yield) as a colourless oil. ¹H NMR (500 MHz, DMSO) δ 7.39 (d, 1H), 6.67 (d, 1H), 5.27 (s, 2H), 3.72 (dd, 2H), 3.28 (dd, 2H), 3.05 (dd, 2H), 0.91-0.85 (m, 2H), −0.05 (s, 9H). LCMS no ionization.

### Step B: Trimethyl-[2-[[3-(4,4,5,5-tetramethyl-1,3,2-dioxaborolan-2-yl)-2-bicyclo[4.2.0]octa-1,3,5-trienyl]oxymethoxy]ethyl]silane

2-[(3-Bromo-2-bicyclo[4.2.0]octa-1,3,5-trienyl)oxymethoxy]ethyl-trimethyl-silane (103.0 mg, 0.270 mmol, 1 eq), bis(pinacolato)diboron (81.0 mg, 0.320 mmol, 1.2 eq) and potassium acetate (111.0 mg, 1.13 mmol, 4.25 eq) in isopropyl acetate (8 mL) was sparged (bubbling nitrogen for 10 min whilst sonicating). XPhos Pd G3 (46.0 mg, 0.05 mmol, 0.05 eq) and XPhos (11.0 mg, 0.02 mmol, 0.02 eq) were added and the reaction mixture was stirred at 90° C. for 16 h. The reaction mixture was concentrated, and the resulting residue was purified by chromatography on silica gel (40 g, 0-20% MTBE:isoHexane) to afford the title compound (199 mg, 41% yield) as a light-yellow oil. ¹H NMR (500 MHz, CDCl₃) δ 7.57 (d, 1H), 6.71 (d, 1H), 5.25 (s, 2H), 3.81-3.71 (m, 2H), 3.30 (dd, 2H), 3.18-3.05 (m, 2H), 1.33 (s, 12H), 0.97-0.92 (m, 2H), −0.03 (s, 9H). LCMS no ionization.

### Step C: N-[(3R)-1-Ethyl-3-piperidyl]-1-methyl-7-[2-(2-trimethylsilylethoxymethoxy)-3-bicyclo[4.2.0]octa-1(6),2,4-trienyl]pyrazolo[3,4-d]pyridazin-4-amine

A mixture of 7-chloro-N-[(3R)-1-ethyl-3-piperidyl]-1-methyl-pyrazolo[3,4-d]pyridazin-4-amine (Example 6, step D) (48 mg, 0.16 mmol, 1.00 eq), aforementioned trimethyl-[2-[[3-(4,4,5,5-tetramethyl-1,3,2-dioxaborolan-2-yl)-2-bicyclo[4.2.0]octa-1,3,5-trienyl]oxymethoxy]ethyl]silane (93 mg, 0.22 mmol, 1.37 eq), potassium carbonate (108 mg, 0.78 mmol, 4.80 eq) and 1,1′-bis(diphenylphosphino)ferrocene-palladium(II) dichloride dichloromethane complex (20 mg, 0.02 mmol, 0.150 eq) in 1,4-dioxane (1.4 mL) and water (0.70 mL) was flushed with argon and stirred at 95° C. for 16 hours. The reaction mixture was extracted with ethyl acetate and water. The aqueous layer was backextracted with ethyl acetate. The organic layers were washed with water and brine. The combined organic layers were dried over sodium sulfate, filtered and concentrated in vacuo. The crude product was adsorbed on ISOLUTE HM-N and purified by flash chromatography (silica gel, 12 g, gradient 0% to 100% (dichloromethane:methanol:NH₄OH 9:1:0.05) in dichloromethane). All fractions containing product were combined and concentrated in vacuo to afford the title compound (33 mg, 38% yield) as a dark green oil. LCMS: m/z 509.4 [M+H]⁺, ESI pos.

### Step D: 3-[4-[[(3R)-1-Ethyl-3-piperidyl]amino]-1-methyl-pyrazolo[3,4-d]pyridazin-7-yl]bicyclo[4.2.0]octa-1(6),2,4-trien-2-ol

To a solution of N-[(3R)-1-ethyl-3-piperidyl]-1-methyl-7-[2-(2-trimethylsilylethoxymethoxy)-3-bicyclo[4.2.0]octa-1(6),2,4-trienyl]pyrazolo[3,4-d]pyridazin-4-amine (Example 18, step A) (33 mg, 0.06 mmol, 1.00 eq) in dichloromethane (1.6 mL) and methanol (0.40 mL) was added dropwise 4 M HCl in dioxane (192 mg, 0.160 mL, 0.64 mmol, 10.39 eq). The reaction mixture was stirred at room temperature for 16 hours. The reaction mixture was diluted with dichloromethane/methanol (19:1), added carefully onto a mixture of 1 mL ice cold water and 4 mL saturated aq. NaHCO₃-solution and then extracted with dichloromethane/methanol (19:1). The organic layer was washed with brine. The aqueous layers were backextracted twice with dichloromethane/methanol (19:1). The combined organic layers were dried over sodium sulfate, filtered and concentrated in vacuo to afford the title compound (25 mg, 96% yield, 90% purity) as a brown foam. LCMS: m/z 379.3 [M+H]⁺, ESI pos.

### Example 19

### (3S,5R)-1-Ethyl-5-[[7-(4-hydroxy-2,3-dihydrobenzofuran-5-yl)-1-methyl-pyrazolo[3,4-d]pyridazin-4-yl]amino]piperidin-3-ol

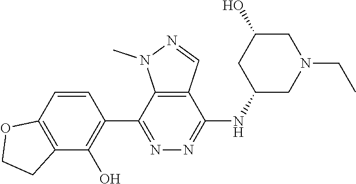 [SMILES: CCN1C[C@@H](O)C[C@@H](Nc2nnc(-c3ccc4c(c3O)CCO4)c3c2cnn3C)C1]

### Step A: tert-Butyl (3R,5S)-3-[(7-chloro-1-methyl-pyrazolo[3,4-d]pyridazin-4-yl)amino]-5-hydroxy-piperidine-1-carboxylate

To a mixture of 4,7-dichloro-1-methyl-pyrazolo[3,4-d]pyridazine (Example 6, step C) (200 mg, 0.94 mmol, 1.00 eq) and tert-butyl (3R,5S)-3-amino-5-hydroxy-piperidine-1-carboxylate (CAS #1932513-59-1, 306 mg, 1.41 mmol, 1.51 eq) in 1,4-dioxane (1.2 mL) was added N,N-diisopropylethylamine (366 mg, 0.494 mL, 2.83 mmol, 3.02 eq). The reaction mixture was stirred at 100° C. for two days and then left standing at room temperature for three days. The reaction mixture was extracted with ethyl acetate and water. The aqueous layer was backextracted with ethyl acetate. The organic layers were washed with water and brine. The combined organic layers were dried over sodium sulfate, filtered and concentrated in vacuo. The residue was adsorbed on ISOLUTE HM-N and purified by flash chromatography (silica gel, 25 g, gradient 0% to 5% methanol in dichloromethane) to afford the title compound (210 mg, 56% yield) as an off-white solid. LCMS: m/z 383.2 [M+H]⁺, ESI pos.

### Step B: (3S,5R)-5-[(7-Chloro-1-methyl-pyrazolo[3,4-d]pyridazin-4-yl)amino]piperidin-3-ol hydrochloride

To a solution of tert-butyl (3R,5S)-3-[(7-chloro-1-methyl-pyrazolo[3,4-d]pyridazin-4-yl)amino]-5-hydroxy-piperidine-1-carboxylate (Example 19, step A) (205 mg, 0.51 mmol, 1.00 eq) in dichloromethane (1.0 mL) and methanol (0.50 mL) was added dropwise 4 M HCl in dioxane (1.2 mL, 4.8 mmol, 9.44 eq). The reaction mixture was stirred at room temperature for 1.5 hours. The reaction mixture was concentrated in vacuo to afford the title compound (162 mg, 95% yield) as an off-white solid. LCMS: m/z 283.2 [M+H]⁺, ESI pos.

### Step C: (3S,5R)-5-[(7-Chloro-1-methyl-pyrazolo[3,4-d]pyridazin-4-yl)amino]-1-ethyl-piperidin-3-ol

To a suspension of (3S,5R)-5-[(7-chloro-1-methyl-pyrazolo[3,4-d]pyridazin-4-yl)amino]piperidin-3-ol hydrochloride (Example 19, step B) (160 mg, 0.48 mmol, 1.00 eq) in dichloromethane (3.0 mL) was added acetaldehyde (47 mg, 0.06 mL, 1.06 mmol, 2.23 eq) followed by sodium acetate (79 mg, 0.96 mmol, 2.02 eq) under ice-bath cooling. Sodium triacetoxyborohydride (152 mg, 0.72 mmol, 1.51 eq) was added in three portions at 0° C. After the addition was complete, the ice-bath was removed and the reaction mixture was stirred at room temperature for 1 hour. The reaction mixture was carefully quenched with saturated aq. NaHCO₃-solution and extracted three times with dichloromethane. The combined organic layers were dried over sodium sulfate, filtered and concentrated in vacuo. The crude product was adsorbed on ISOLUTE HM-N and purified by flash chromatography (silica gel, 12 g, gradient 0% to 10% methanol in dichloromethane) to afford the title compound (102 mg, 65% yield) as a light yellow foam. LCMS: m/z 311.2 [M+H]⁺, ESI pos.

### Step D: (3S,5R)-1-Ethyl-5-[[7-(4-hydroxy-2,3-dihydrobenzofuran-5-yl)-1-methyl-pyrazolo[3,4-d]pyridazin-4-yl]amino]piperidin-3-ol

A mixture of (3S,5R)-5-[(7-chloro-1-methyl-pyrazolo[3,4-d]pyridazin-4-yl)amino]-1-ethyl-piperidin-3-ol (Example 19, step C) (90 mg, 0.28 mmol, 1.00 eq), 5-(4,4,5,5-tetramethyl-1,3,2-dioxaborolan-2-yl)-2,3-dihydrobenzofuran-4-ol (Example 17, step C) (140 mg, 0.51 mmol, 1.84 eq), potassium carbonate (170 mg, 1.23 mmol, 4.47 eq) and 1,1′-bis(diphenylphosphino)ferrocene-palladium(II) dichloride dichloromethane complex (34 mg, 0.04 mmol, 0.15 eq) in 1,4-dioxane (1.6 mL) and water (0.80 mL) was flushed with argon and stirred at 95° C. for 16 hours. The reaction mixture was extracted with ethyl acetate and half-saturated aq. NH₄Cl-solution. The aqueous layer was backextracted with ethyl acetate. The organic layers were washed with water and brine. The combined organic layers were dried over sodium sulfate, filtered and concentrated in vacuo. The crude product was adsorbed on ISOLUTE HM-N and purified by flash chromatography (silica gel, 12 g, gradient 0% to 100% (dichloromethane:methanol:NH₄OH 9:1:0.05) in dichloromethane). All fractions containing product were combined and concentrated in vacuo to afford the title compound (58 mg, 49% yield) as a brown foam. LCMS: m/z 411.3 [M+H]⁺, ESI pos.

### Example A

A compound of formula I can be used in a manner known per se as the active ingredient for the production of tablets of the following composition:

| | Per tablet | |
|---|---|---|
| Active ingredient | 200 | mg |
| Microcrystalline cellulose | 155 | mg |
| Corn starch | 25 | mg |
| Talc | 25 | mg |
| Hydroxypropylmethylcellulose | 20 | mg |
| | 425 | mg |

### Example B

A compound of formula I can be used in a manner known per se as the active ingredient for the production of capsules of the following composition:

| | Per capsule | |
|---|---|---|
| Active ingredient | 100.0 | mg |
| Corn starch | 20.0 | mg |
| Lactose | 95.0 | mg |
| Talc | 4.5 | mg |
| Magnesium stearate | 0.5 | mg |
| | 220.0 | mg |

### Example A′

A compound of formula Ib can be used in a manner known per se as the active ingredient for the production of tablets of the following composition:

| | Per tablet | |
|---|---|---|
| Active ingredient | 200 | mg |
| Microcrystalline cellulose | 155 | mg |
| Corn starch | 25 | mg |
| Talc | 25 | mg |
| Hydroxypropylmethylcellulose | 20 | mg |
| | 425 | mg |

### Example B′

A compound of formula Ib can be used in a manner known per se as the active ingredient for the production of capsules of the following composition:

| | Per capsule | |
|---|---|---|
| Active ingredient | 100.0 | mg |
| Corn starch | 20.0 | mg |
| Lactose | 95.0 | mg |
| Talc | 4.5 | mg |
| Magnesium stearate | 0.5 | mg |
| | 220.0 | mg |

1. Compounds of formula I 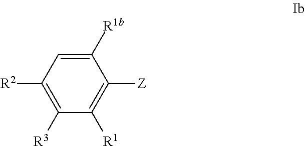
wherein
R¹ is H, alkoxy, haloalkyl or OH;
R^{1b} is H, halo or alkyl;
R² is halo, haloalkyl, haloalkoxy, nitrile or alkyl;
R³ is H;
or R² and R³, and the atoms to which they are attached, bond together to form either a heterocycle comprising 1 O heteroatom or a cycloalkyl ring;
Z is selected from ring-systems 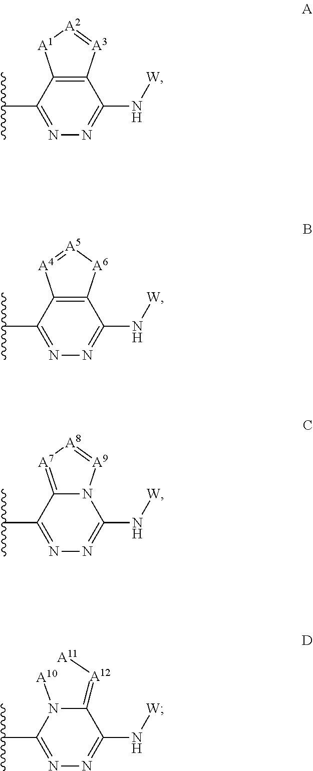
A¹ is S, NR^{X1} or O, wherein R^{X1} is H, alkyl, or cyclopropyl;
A² is CR^{Y1} or N, wherein R^{Y1} is H or alkyl;
A³ is CR^{Z1} or N, wherein R^{Z1} is H or alkyl; wherein if A¹ is S or O then A² and A³ cannot both be N;
A⁴ is CR^{Z2} or N, wherein R^{Z2} is H or alkyl;
A⁵ is CR^{Y2} or N, wherein CR^{Y2} is H or alkyl;
A⁶ is S, NR^{X2} or O, wherein R^{X2} is H or alkyl; wherein if A⁶ is S or O then A⁴ and A⁵ cannot both be N;
A⁷, A⁸ and A⁹ are independently CR^{W1} or N, wherein CR^{W1} is H or alkyl; wherein A⁷, A⁸ and A⁹ cannot be all N;
A¹⁰, A¹¹ and A¹² are independently CR^{W2} or N, wherein CR^{W2} is H or alkyl; wherein A¹⁰, A¹¹ and A¹² cannot be all N;
W is a substituted 4-member-cycloalkyl, a substituted 6-member-cycloalkyl, a substituted 6-member-heterocycle comprising a single heteroatom N, or 1,2,3,5,6,7,8,8a-octahydroindolizin-7-yl, wherein substituted 4-member-cycloalkyl is substituted with hydroxyl and methyl, substituted 6-member-cycloalkyl is substituted with OH, and substituted 6-member-heterocycle comprising a single heteroatom N is substituted with one or two substituents independently selected from alkyl, OH or halo;
and pharmaceutically acceptable salts.

2. A compound according to claim 1, wherein
Z is selected from
Ring System A, wherein
A¹ is S, NR^{X1} or O, wherein R^{X1} is H or alkyl;
A² is CR^{Y1} or N, wherein R^{Y1} is H;
A³ is CR^{Z1} or N, wherein R^{Z1} is H or alkyl;
wherein if A¹ is S or O then neither A² nor A³ can be N; or
Ring System B, wherein
A⁴ is CR^{Z2}, wherein R^{Z2} is H;
A⁵ is CR^{Y2} or N, wherein CR^{Y2} is H;
A⁶ is S or NR^{X2}, wherein R^{X2} is alkyl;
wherein if A⁶ is S then neither A⁴ nor A⁵ can be N.

3. A compound according to claim 1, wherein Z is Ring System A, wherein
A¹ is S, NR^{X1} or O, wherein R^{X1} is H or alkyl;
A² is CR^{Y1} or N, wherein R^{Y1} is H;
A³ is CR^{Z1} or N, wherein R^{Z1} is H or alkyl;
wherein if A¹ is S or O then neither A² nor A³ can be N.

4. A compound according to claim 1, wherein Ring System A comprises 2 N heteroatoms.

5. A compound according to claim 1, wherein Z is Ring System A, wherein
A¹ is NR^{X1}, wherein R^{X1} is alkyl;
A² is N; and
A³ is CR^{Z1}, wherein R^{Z1} is H.

6. A compound according to claim 1, wherein R¹ is H or OH.

7. A compound according to claim 1, wherein R¹ is OH.

8. A compound according to claim 1, wherein R^{1b} is H.

9. A compound according to claim 1, wherein R² is halo, haloalkyl, or haloalkoxy and R³ is H; or
R² and R³, and the atoms to which they are attached, bond together to form either a heterocycle comprising 1 O heteroatom or a 4-member cycloalkyl ring.

10. A compound according to claim 1, wherein R² and R³, and the atoms to which they are attached, bond together to form either a heterocycle comprising 1 O heteroatom or a cycloalkyl ring.

11. A compound according to claim 1, wherein R² and R³, and the atoms to which they are attached, bond together to form a cycloalkyl ring.

12. A compound according to claim 1, wherein R² and R³, and the atoms to which they are attached, bond together to form either a 5-member heterocycle comprising 1 O heteroatom or a 4-member cycloalkyl ring.

13. A compound according to claim 1, wherein R² and R³, and the atoms to which they are attached, bond together to form a 4-member cycloalkyl ring.

14. A compound according to claim 1, wherein W is a substituted 4-member-cycloalkyl, a substituted 6-member-cycloalkyl, or a substituted 6-member-heterocycle comprising a single heteroatom N, wherein substituted 4-member-cycloalkyl is substituted with hydroxyl and methyl, substituted 6-member-cycloalkyl is substituted with OH, and substituted 6-member-heterocycle comprising a single heteroatom N is substituted with one or two substituents independently selected from alkyl and OH.

15. A compound according to claim 1, wherein W is ethylpiperidyl or 1-ethyl-piperidin-3-ol.

16. A compound according to claim 1, wherein
W is ethylpiperidyl.

17. A compound according to claim 1, wherein
R¹ is H or OH;
R^{1b} is H, halo or alkyl;
R² is halo, haloalkyl or haloalkoxy;
R³ is H;
or R² and R³, and the atoms to which they are attached, bond together to form either a 5-member heterocycle comprising 1 O heteroatom or a 4-member cycloalkyl ring;
Z is selected from
Ring System A, wherein
A¹ is S, NR^{X1} or O, wherein R^{X1} is H or alkyl;
A² is CR^{Y1} or N, wherein R^{Y1} is H;
A³ is CR^{Z1} or N, wherein R^{Z1} is H or alkyl;
wherein if A¹ is S or O then neither A² nor A³ can be N; or
Ring System B, wherein
A⁴ is CR^{Z2}, wherein R^{Z2} is H;
A⁵ is CR^{Y2} or N, wherein CR^{Y2} is H;
A⁶ is S or NR^{X2}, wherein R^{X2} is alkyl;
wherein if A⁶ is S then neither A⁴ nor A⁵ can be N;
W is a substituted 4-member-cycloalkyl, a substituted 6-member-cycloalkyl, or a substituted 6-member-heterocycle comprising a single heteroatom N, wherein substituted 4-member-cycloalkyl is substituted with hydroxyl and methyl, substituted 6-member-cycloalkyl is substituted with OH, and substituted 6-member-heterocycle comprising a single heteroatom N is substituted with one or two substituents independently selected from alkyl and OH;
and pharmaceutical acceptable salts thereof.

18. A compound according to claim 1, wherein
R¹ is H or OH;
R^{1b} is H, halo or alkyl;
R² is halo, haloalkyl or haloalkoxy;
R³ is H;
or R² and R³, and the atoms to which they are attached, bond together to form either a 5-member heterocycle comprising 1 O heteroatom or a 4-member cycloalkyl ring;
Z is Ring System A, wherein
A¹ is S, NR^{X1} or O, wherein R^{X1} is H or alkyl;
A² is CR^{Y1} or N, wherein R^{Y1} is H;
A³ is CR^{Z1} or N, wherein R^{Z1} is H or alkyl;
wherein if A¹ is S or O then neither A² nor A³ can be N;
W is a substituted 4-member-cycloalkyl, a substituted 6-member-cycloalkyl, or a substituted 6-member-heterocycle comprising a single heteroatom N, wherein substituted 4-member-cycloalkyl is substituted with hydroxyl and methyl, substituted 6-member-cycloalkyl is substituted with OH, and substituted 6-member-heterocycle comprising a single heteroatom N is substituted with one or two substituents independently selected from alkyl and OH;
and pharmaceutical acceptable salts thereof.

19. A compound according to claim 1, wherein
R¹ is OH;
R^{1b} is H;
R² and R³, and the atoms to which they are attached, bond together to form either a 5-member heterocycle comprising 1 O heteroatom or a 4-member cycloalkyl ring;
Z is Ring System A, wherein
A¹ is NR^{X1}, wherein R^{X1} is alkyl;
A² is N;
A³ is CR^{Z1}, wherein R^{Z1} is H;
W is ethylpiperidyl or 1-ethyl-piperidin-3-ol;
and pharmaceutical acceptable salts thereof.

20. A compound according to claim 1, wherein
R¹ is OH;
R^{1b} is H;
R² and R³, and the atoms to which they are attached, bond together to form either a 4-member cycloalkyl ring;
Z is Ring System A, wherein
A¹ is NR^{X1}, wherein R^{X1} is alkyl;
A² is N;
A³ is CR^{Z1}, wherein R^{Z1} is H;
W is ethylpiperidyl;
and pharmaceutical acceptable salts thereof.

21. A compound according to claim 1, selected from
(rac)-2-[7-[(1-Ethyl-3-piperidyl)amino]thieno[2,3-d]pyridazin-4-yl]-5(trifluoromethyl)phenol; formic acid;
(rac)-2-[7-[(1-Ethyl-3-piperidyl)amino]thieno[2,3-d]pyridazin-4-yl]-5(trifluoromethyl)phenol;
(rac)-2-[4-[(1-Ethyl-3-piperidyl)amino]thieno[2,3-d]pyridazin-7-yl]-5-(trifluoromethyl)phenol; formic acid;
(rac)-2-[4-[(1-Ethyl-3-piperidyl)amino]thieno[2,3-d]pyridazin-7-yl]-5-(trifluoromethyl)phenol;
(rac)-N-(1-ethyl-3-piperidyl)-4-[4-(trifluoromethyl)phenyl]thieno[2,3-d]pyridazin-7-amine;
(rac)-N-(1-ethyl-3-piperidyl)-7-[4-(trifluoromethyl)phenyl]thieno[2,3-d]pyridazin-4-amine;
2-[4-[[(3R)-1-Ethyl-3-piperidyl]amino]-1-methyl-pyrrolo[2,3-d]pyridazin-7-yl]-5-(trifluoromethyl)phenol; hydrochloride;
2-[4-[[(3R)-1-ethyl-3-piperidyl]amino]-1-methyl-pyrrolo[2,3-d]pyridazin-7-yl]-5-(trifluoromethyl)phenol; 2,2,2-trifluoroacetic acid;
2-[4-[[(3R)-1-ethyl-3-piperidyl]amino]-1-methyl-pyrrolo[2,3-d]pyridazin-7-yl]-5-(trifluoromethyl)phenol;
2-[4-[[(3R)-1-Ethyl-3-piperidyl]amino]-1-methyl-pyrazolo[3,4-d]pyridazin-7-yl]-5-(trifluoromethyl)phenol; 2,2,2-trifluoroacetic acid;
2-[4-[[(3R)-1-Ethyl-3-piperidyl]amino]-1-methyl-pyrazolo[3,4-d]pyridazin-7-yl]-5-(trifluoromethyl)phenol;
2-[4-[[(3R)-1-ethyl-3-piperidyl]amino]furo[2,3-d]pyridazin-7-yl]-5-(trifluoromethyl)phenol;
2-[4-[[(3R)-1-ethyl-3-piperidyl]amino]-1-methyl-imidazo[4,5-d]pyridazin-7-yl]-5-(trifluoromethyl)phenol;
2-[7-[[(3R)-1-ethyl-3-piperidyl]amino]-1-methyl-pyrazolo[3,4-d]pyridazin-4-yl]-5-(trifluoromethyl)phenol;
2-[4-[[(3R)-1-ethyl-3-piperidyl]amino]-1-methyl-pyrazolo[3,4-d]pyridazin-7-yl]-5-(trifluoromethoxy)phenol;
2-[4-[[(1R,2R)-2-hydroxycyclohexyl]amino]-1-methyl-pyrazolo[3,4-d]pyridazin-7-yl]-5-(trifluoromethyl)phenol;
5-Chloro-2-[1-methyl-4-[[(3R)-1-ethyl-3-piperidyl]amino]pyrazolo[3,4-d]pyridazin-7-yl]phenol;
2-[4-[[(3R)-1-ethyl-3-piperidyl]amino]-3-methyl-isoxazolo[4,5-d]pyridazin-7-yl]-5-(trifluoromethyl)phenol;
2-[4-[[(3R)-1-ethyl-3-piperidyl]amino]-1H-pyrazolo[3,4-d]pyridazin-7-yl]-3-methyl-5-(trifluoromethyl)phenol;
2-[4-[[(3R)-1-ethyl-3-piperidyl]amino]-1-methyl-pyrazolo[3,4-d]pyridazin-7-yl]-3-fluoro-5-(trifluoromethyl)phenol;
2-[4-[(3-hydroxy-3-methyl-cyclobutyl)amino]-1-methyl-pyrazolo[3,4-d]pyridazin-7-yl]-5-(trifluoromethyl)phenol;
5-[4-[[(3R)-1-Ethyl-3-piperidyl]amino]-1-methyl-pyrazolo[3,4-d]pyridazin-7-yl]-2,3-dihydrobenzofuran-4-ol;
3-[4-[[(3R)-1-ethyl-3-piperidyl]amino]-1-methyl-pyrazolo[3,4-d]pyridazin-7-yl]bicyclo[4.2.0]octa-1(6),2,4-trien-2-ol;
(3S,5R)-1-Ethyl-5-[[7-(4-hydroxy-2,3-dihydrobenzofuran-5-yl)-1-methyl-pyrazolo[3,4-d]pyridazin-4-yl]amino]piperidin-3-ol;
and pharmaceutically acceptable salts thereof.

22. (canceled)

23. (canceled)

24. A compound according to claim 1, wherein the compound is 3-[4-[[(3R)-1-ethyl-3-piperidyl]amino]-1-methyl-pyrazolo[3,4-d]pyridazin-7-yl]bicyclo[4.2.0]octa-1(6),2,4-trien-2-ol, and pharmaceutically acceptable salts thereof.

25. (canceled)

26. (canceled)

27. A pharmaceutical composition comprising a compound according to claim 1 and a therapeutically inert carrier.

28-32. (canceled)

33. A method of inhibiting NLRP3, which method comprises administering an effective amount of a compound as claimed in claim 1 to inhibit NLRP3.

34. A method for the treatment or prophylaxis of a disease, disorder or condition, which method comprises administering an effective amount of a compound according to claim 1, wherein the disease, disorder or condition is selected from Asthma or COPD.

35. A method for the treatment or prophylaxis of a disease, disorder or condition, which method comprises administering an effective amount of a compound according to claim 1, wherein the disease, disorder or condition is selected from Parkinson's Disease or Alzheimer's Disease.

36. (canceled)
